(19) United States
(12) Patent Application Publication
(10) Pub. No.: US 2023/0105342 A1
(43) Pub. Date: Apr. 6, 2023

(54) METHOD FOR PRODUCING RECOMBINANT PROTEINS IN INSECTS

(71) Applicant: Future Fields Cellular Agriculture and Research LTD., Edmonton (CA)

(72) Inventor: Matthew Anderson-Baron, Edmonton (CA)

(21) Appl. No.: 18/075,362

(22) Filed: Dec. 5, 2022

Related U.S. Application Data
(63) Continuation-in-part of application No. 17/086,226, filed on Oct. 30, 2020.
(60) Provisional application No. 63/285,938, filed on Dec. 3, 2021.
(60) Provisional application No. 62/927,788, filed on Oct. 30, 2019.

(51) Int. Cl. C12N 15/85; A01K 67/033

(52) U.S. Cl. CPC C12N 15/8509; A01K 67/0339; C12N 2015/8518; A01K 2267/01; A01K 2227/706; A01K 2217/052

(57) ABSTRACT

One variation of a method includes: during an initial period, modifying a population of insects to produce a target compound responsive to application of a stressor; during a growth period succeeding the initial period, cultivating the population of insects according to a set of growth conditions; and, during a treatment period succeeding the growth period, applying a dosage of the stressor to the population of insects to trigger production of the target compound. The method further includes, during a harvest period succeeding the treatment period: harvesting the population of insects; homogenizing the population of insects to form a blend including a set of secondary components and an amount of the target compound; extracting the amount of the target compound from the blend; and mixing the first amount of the first target compound with a set of stabilizing agents configured to stabilize the target compound.

### CROSS-REFERENCE TO RELATED APPLICATIONS

This application claims the benefit of U.S. Provisional Application No. 63/285,938, filed on 3 Dec. 2021, which is incorporated in its entirety by this reference.

This application is a continuation-in-part of U.S. patent application Ser. No. 17/086,226, filed on 30 Oct. 2020, which claims the benefit of U.S. Provisional Application No. 62/927,788, filed on 30 Oct. 2019, each of which is incorporated in its entirety by this reference.

### TECHNICAL FIELD

This invention relates generally to the field of cellular agriculture and more specifically to a new and useful method for producing recombinant proteins in the field of cellular agriculture.

### BRIEF DESCRIPTION OF THE FIGURES

FIG. 1 is a flowchart representation of a method;

FIG. 2 is a flowchart representation of the method;

FIG. 3 is a flowchart representation of the method;

FIG. 4 is a flowchart representation of the method;

FIG. 5 is a flowchart representation of the method;

FIG. 6 is a flowchart representation of the method; and

FIG. 7 is a flowchart representation of the method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### BACKGROUND OF THE INVENTION

Genetic engineering techniques have been developed to enable expression of exogenous genes in organisms. For example, U.S. Pat. No. 5,472,858 describes genetic techniques that enable recombinant protein expression in insects. U.S. Patent Publication No. 2012/0003715, W.O. Patent Pub. No. 2018/225917, and W.O. Pat. Pub. No. 2002/006464 similarly describe recombinant protein expression in insects. Further, Bachmann et al (2008) describes genetic techniques that enable integration of exogenous genes in flies. Further, genetic techniques have been developed to enable inducible gene expression. For example, Akmammedov et al (2017) describes systems for temporal gene expression based on various promoters and inducers (e.g., stressors). In another example, Malkeyeva et al (2020) describes a cold-stress response in Drosophila.

The present invention addresses a need for methods and techniques that enable large-scale production of recombinant proteins via an inducible, gene-expression system configured to transiently induce expression of recombinant proteins in genetically-modified insect populations in order to yield quantities of recombinant proteins compatible for integration in various applications, such as food (e.g., cultured foods), cosmetics, vaccine, diagnostics, therapeutics, etc.

### 1. Method

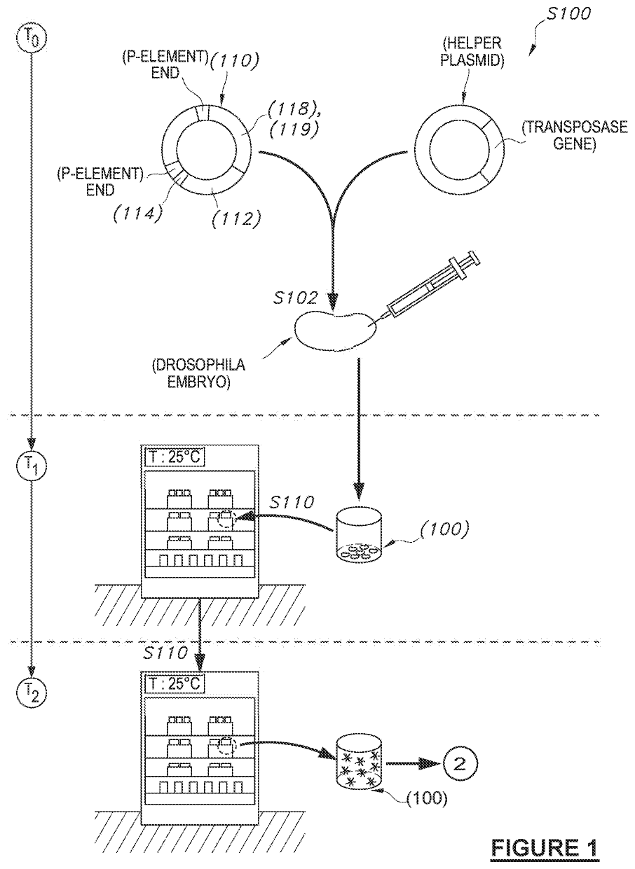

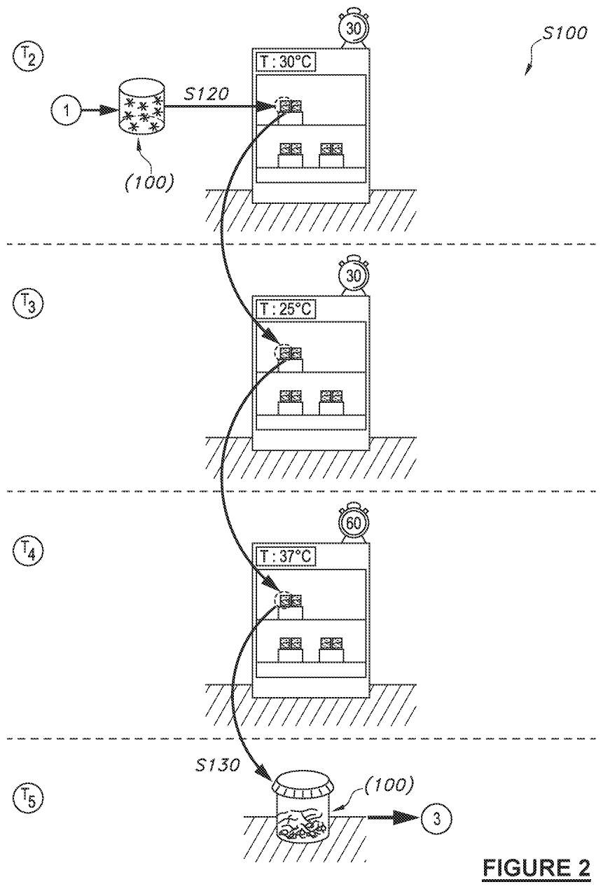

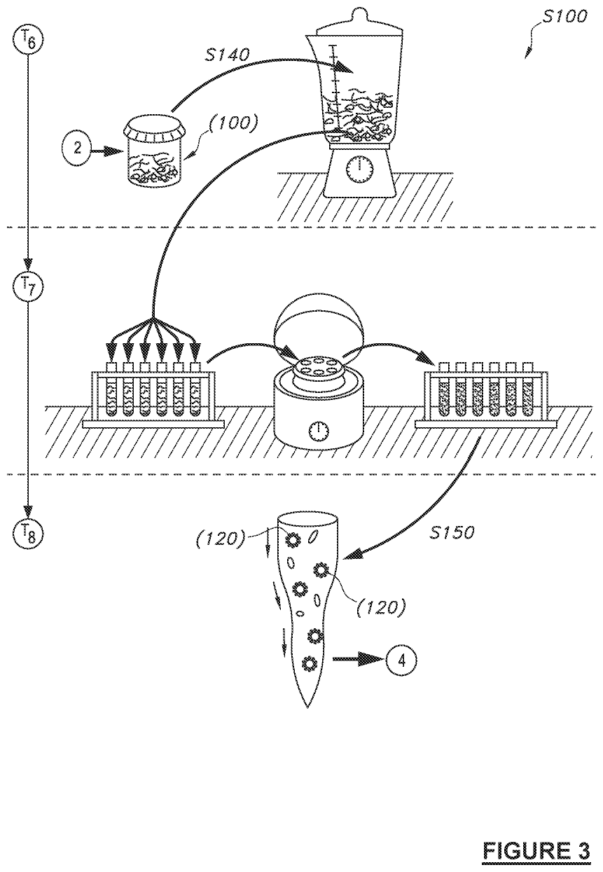

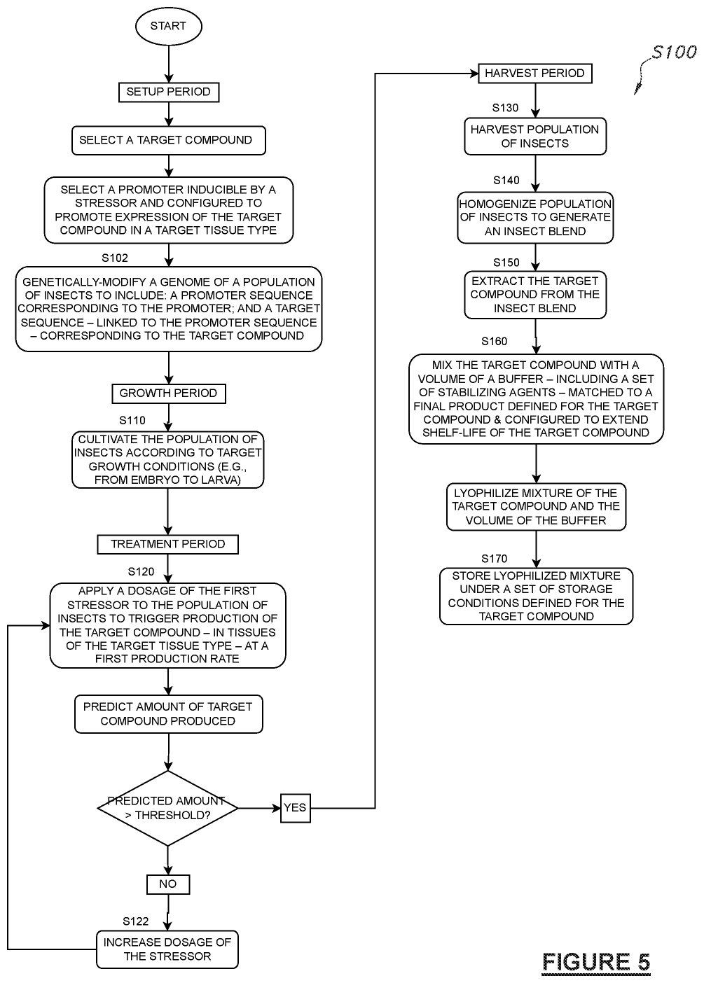

As shown in FIGS. 1-7, a method S100 includes: during an initial period, genetically modifying a population of Drosophila to produce a first target compound 120 responsive to exposure to a heat stressor in Block S102, a genome of the population of Drosophila including a first promoter sequence 114, configured to activate responsive to exposure to the heat stressor, and a first target sequence 112 linked to the first promoter sequence 114 and corresponding to the first target compound 120; and, during a growth period succeeding the initial period, cultivating the population of Drosophila for a first duration under a first set of growth conditions in Block S110. The method S100 further includes, during a treatment period succeeding the growth period: during a first treatment cycle within the treatment period, applying a first dosage of the heat stressor to the population of Drosophila to trigger production of the first target compound 120 in the population of Drosophila at a first rate proportional the first dosage in Block S120; during a second treatment cycle succeeding the first treatment cycle within the treatment period, applying a second dosage of the heat stressor, the second dosage greater than the first dosage, to the population of Drosophila to trigger production of the first target compound 120 at a second rate greater than the first rate and proportional the second dosage in Block S122; and, based on a first predicted amount of the first target compound 120 produced in the population of Drosophila during the treatment period, harvesting the population of Drosophila in Block S130. The method S100 further includes, during a purification period succeeding the treatment period, in response to harvesting the population of Drosophila: homogenizing the population of Drosophila to generate a blend including secondary components and a first amount of the first target compound 120 in Block S140; and extracting the first amount of the first target compound 120 from the blend in Block S150.

One variation of the method S100 includes: during an initial period, modifying a population of Drosophila to produce a first target compound 120, in a set of target compound 120S, a genome of the population of Drosophila including a promoter sequence 114, inducible by a first stressor and associated with cell tissue of a first tissue type, and a first target sequence 112 coupled to the first promoter sequence 114 and corresponding to the first target compound 120 in Block S102. In this variation, the method S100 further includes: during a growth period succeeding the initial period, cultivating the population of Drosophila according to a set of growth conditions in Block S110; and, during a treatment period succeeding the growth period, applying a first dosage of the first stressor to the population of Drosophila to activate the first promoter sequence 114 and trigger production of the first target compound 120 in tissue of the first tissue type in the population of Drosophila in Block S120. In this variation, the method S100 further includes, during a harvest period succeeding the treatment period: harvesting the population of Drosophila in Block S130; homogenizing the population of Drosophila to form a blend including a set of secondary components and a first amount of the first target compound 120 in Block S140; extracting the first amount of the first target compound 120 from the blend in Block S150; and mixing the first amount of the first target compound 120 with a set of stabilizing agents configured to stabilize the first target compound 120 to generate a compound mixture in Block S160. The method S100 further includes, during a storage period, storing the compound mixture according to a set of storage conditions defined for the first target compound 120 in Block S170.

One variation of the method S100 includes, during a first production cycle for a first population of Drosophila genetically-modified to produce a first target compound 120: during a first growth period, cultivating the first population of Drosophila according to a first set of growth conditions in Block S110, a first genome of the first population of Drosophila including a first promoter sequence 114 associated with a first stressor and corresponding to a first set of target characteristics defined for the first target compound 120, and a first target sequence 112 coupled to the first promoter sequence 114 and corresponding to the first target compound 120; and, during a first treatment period succeeding the first growth period, applying a first dosage of the first stressor to the population of Drosophila to activate the first promoter sequence 114 and trigger production of the first target compound 120 in the first population of Drosophila in Block S120. The method S100 further includes, during a first harvest period, succeeding the first treatment period, within the first production cycle: harvesting the first population of Drosophila in Block S130; homogenizing the first population of Drosophila to form a first blend including a first set of secondary components and a first amount of the first target compound 120 in Block S140; and extracting the first amount of the first target compound 120, exhibiting the first set of target characteristics, from the first blend in Block S150.

In the preceding variation, the method S100 further includes, during a second production cycle for a second population of Drosophila genetically-modified to produce a second target compound 120: during a second growth period in Block S110, cultivating the second population of Drosophila according to a second set of growth conditions, a second genome of the second population of Drosophila including a second promoter sequence 114 associated with a second stressor and corresponding to a second set of target characteristics defined for the second target compound 120, and a second target sequence 112 coupled to the second promoter sequence 114 and corresponding to the second target compound 120; and, during a second treatment period succeeding the second growth period, applying a second dosage of the second stressor to the population of Drosophila to activate the second promoter sequence 114 and trigger production of the second target compound 120 in the second population of Drosophila in Block S120. In this variation, the method S100 further includes, during a second harvest period succeeding the second treatment period: harvesting the second population of Drosophila in Block S130; homogenizing the second population of Drosophila to form a second blend including a second set of secondary components and a second amount of the second target compound 120 in Block S140; and extracting the second amount of the second target compound 120, exhibiting the second set of target characteristics, from the second blend in Block S150.

As shown in FIGS. 1-7, one variation of the method S100 for producing a target compound 120 includes: during an initial period, genetically modifying a population of insects 100 to produce a first target compound 120 in Block S102; during a growth period succeeding the initial period, cultivating the population of insects 100 according to a set of growth conditions in Block S110; and, during a treatment period succeeding the growth period, applying a first dosage of a first stressor to the population of insects 100, the first stressor configured to trigger production of the target compound 120 by the population of insects 100 in Block S120. The method S100 further includes, in response to a proportion of the first target compound 120 within the population of insects 100 exceeding a threshold proportion: harvesting the population of insects 100 in Block S130; homogenizing the population of insects 100 to form a blend including the proportion of the first target compound 120 and a proportion of a set of secondary components in Block S140; and separating the proportion of the first target compound 120 from the second proportion of the set of secondary components for extraction of the proportion of the target compound 120 from the blend in Block S150.

In one variation, genetically modifying the population of insects 100 to produce the first target compound 120 includes genetically modifying a genome of the population of insects 100 to include: a target sequence 112 encoding for the target compound 120; and a promoter sequence 114 coupled to the target sequence 112 and associated with the first stressor.

One variation of the method S100 includes: during a growth period, cultivating a population of insects 100, from a first insect stage to a second insect stage, under a set of growth conditions in Block S110, the population of insects 100 genetically modified to produce a first target compound 120; and, during a treatment period succeeding the growth period, applying a first dosage of a first stressor to the population of insects 100, the first stressor configured to trigger production of the first target compound 120 by the population of insects 100 in Block S120. In this variation, the method S100 further includes, in response to a proportion of the first target compound 120 within the population of insects 100 exceeding a threshold proportion: harvesting the population of insects 100 in Block S130; homogenizing the population of insects 100 to form a blend including the proportion of the first target compound 120 and a proportion of a set of secondary components in Block S140; and separating the proportion of the first target compound 120 from the second proportion of the set of secondary components for extraction of the proportion of the target compound 120 from the blend in Block S150.

One variation of the method S100 includes: during an initial period, genetically modifying a population of insects 100 to produce a first target compound 120 in Block S102; during a growth period succeeding the initial period, cultivating the population of insects 100 for a first duration under a first set of growth conditions in Block S110. The method S100 further includes, during a treatment period succeeding the growth period: applying a first dosage of a first stressor to the population of insects 100 to trigger production of the first target compound 120 at a first rate over a first treatment cycle in Block S120; and applying a second dosage of the first stressor, greater than the first dosage, to the population of insects 100 to trigger production of the target compound 120 at a second rate, greater than the first rate, over a second treatment cycle succeeding the first treatment cycle in Block S124; and, in response to a proportion of the first target compound 120 exceeding a threshold proportion during the treatment period, harvesting the population of insects 100 in Block S130. The method S100 further includes, during a purification period succeeding the treatment period: homogenizing the population of insects 100 to form a blend including the proportion of the first target compound 120 in Block S140; and extracting the proportion of the first target compound 120 from the blend in Block S150.

### 2. Applications

Generally, as shown in FIGS. 1-6, Blocks of the method S100 can be executed: to cultivate a genetically-modified population of insects 100 configured to generate a recombinant protein (or “target compound 120” 120); to control generation of this target compound 120 within the population of insects 100 via application of a particular stressor (e.g., an environmental condition, a chemical agent, a biological agent)—such as heat-shock, cold-shock, UV-exposure, and/or nutrient-deficiency—configured to trigger a specific stress response, in the population of insects 100, linked to generation of the target compound 120; and to extract and purify the target compound 120 from the population of insects 100.

More specifically, a genome of a population of insects 100 (e.g., a population of Drosophila) can be genetically-modified to include: a target sequence 112 encoding for the target compound 120; a promoter sequence 114 coupled to the target compound 120 and associated with the particular stressor, such that application of the particular stressor to the population of insects 100 enables activation of the promoter sequence 114, thereby activating transcription of the downstream target sequence 112 114, leading to expression and therefore production of the target compound 120. Therefore, the method S100 can be executed to induce production of large quantities of the target compound 120 via the genetically-modified population of insects 100.

Further, application of the stressor can be leveraged to both: increase expression of the target sequence 112 via pairing of the target sequence 112 with the promoter sequence 114 associated with the stressor; and decrease expression of other genes present in the genome coupled with the promoter sequence 114. For example, a heat-shock promoter sequence 114 (or “HSP70 promoter sequence 114” 114) can be coupled with a target sequence 112 within the genome of the population of insects 100. When a heat-shock stressor is then applied to this population of insects 100, the heat-shock promoter sequence 114 is activated and promotes transcription of the downstream target sequence 112. However, other genes in the genome that do not include the heat-shock-promoter sequence 114 may exhibit severely inhibited transcription. In addition, when the heat-shock stressor is applied, global translation is substantially halted. However, a leader sequence 116, defining a 5′untranslated region (or “5′UTR”) of heat-shock proteins, can enable translation of heat-shock proteins during application of the stressor, while global translation is halted. In this example, the 5′UTR leader sequence 116 of heat-shock promoter sequence 114 can be included between the heat-shock promoter sequence 114 and the downstream target sequence 112 to enable translation of both heat-shock proteins and the target compound 120. Therefore, in this example, the heat-shock promoter sequence 114 can cooperate with the downstream target sequence 112 to increase enrichment of the target compound 120 encoded by the target sequence 112 within the population of insects 100.

By subjecting insects to conditions configured to induce production of the target compound 120, the population of insects 100 can function as a bioreactor configured to produce large quantities of the target compound 120. Unlike traditional bacterial systems implemented for production of recombinant proteins, the method S100 can be implemented to generate biologically active recombinant proteins, as propagation of the target sequence 112 within the population of insects 100 enables post-translational modifications, such as glycosylation. Further, once the genetically-modified genome is introduced into the population of insects 100, the genome can be propagated through each succeeding generation, requiring minimal maintenance for continued propagation of the genome. Additionally, a size of the population can be scaled accordingly to increase or decrease a quantity of the target compound 120 output by the population of insects 100. Therefore, the target compound 120 can be generated by the population of insects 100 via a scalable, efficient (e.g., high enrichment of the target compound 120 within the population of insects 100), and relatively lower-cost process. In one implementation, the population of insects 100 is a population of Drosophila (or “small fruit flies”).

In one implementation, target compound 120S 120 extracted from the genetically-modified population of insects 100 can be incorporated as growth factors for generation of a growth media configured for cultured meat production (i.e., edible meat produced via growth of stem cells in culture as opposed to harvested from a slaughtered animal) Traditional processes for developing culture media for cultured meat products include supplementing the culture media with fetal bovine serum (or “FBS”) including a mixture of nutrients, growth factors, hormones, lipids, and other components that support cell growth in culture. However, FBS is harvested from fetuses of pregnant cows prior to slaughter, thus resulting in ethical concerns regarding the production of cultured meat products. In addition, costs associated with FBS are increasingly high, resulting in slower growth of the cultured meat market. Conversely, the method S100 can be implemented to genetically-modify a population of insects 100 to produce a set of growth factors which can be mixed with a basal media (e.g., including salts, sugars, amino acids, vitamins) to generate a cost-effective, ethical, serum-free growth media capable of supporting cell growth in culture.

Further, in another implementation, in addition to cultured meat products, the growth factors generated by and extracted from the population of insects 100 can be configured for generation of growth media for production of cultured dairy products (e.g., cultured milk, cultured cheese, cultured ice cream). In yet another implementation, these growth factors can be configured for generation of cultured leather products.

For example, the method S100 can be executed to produce a set of growth factors (i.e., a set of target compound 120S 120), such as FGF2, TGF, IGF1, and/or transferrin. The growth factors produced can be mixed into a growth factor cocktail that supplements a particular basal media to develop a species-specific serum-free growth media. Additionally or alternatively, each growth factor in the set of growth factors can be included in a single “complete” serum-free growth media configured for culturing myoblast and myocyte cell lines, as well as mesenchymal and/or induced-pluripotent stem cell lines. Additionally or alternatively, different combinations of these growth factors, in the set of growth factors, can be included in different batches of the serum-free growth media, enabling customization of serum-free growth media based on a particular product and/or research.

In other various implementations, the method S100 can be executed to efficiently (e.g., high throughput, low cost) produce large quantities of a set of recombinant proteins (i.e., a set of target compound 120S 120) for implementation in development of vaccines, diagnostics, cosmetics, and/or therapeutics. For example, recombinant proteins generated by the population of insects 100 can include Cholera toxin B and/or collagen.

The method S100 is generally described below as executed to produce a target compound 120 in a genetically-modified population of insects 100, such as a population of Drosophila. However, the method S100 can be executed to produce a target compound 120 in genetically-modified populations of insects 100 of various insect types, such as in a genetically-modified population of Drosophila, Diptera, lepidoptera, Cicadida, or mosquitos.

### 3. Growth Factors and Growth Media Production

In one implementation, the target compound 120 can define a growth factor configured for production of a growth media 130 for growing cells in culture, such as myoblasts and/or myoblast derivatives, mesenchymal stem cells, pluripotent stem cells (e.g., induced pluripotent stem cells), etc. In this implementation, the growth factor (i.e., the target compound 120) can be collected from a population of insects 100 according to methods and techniques described below and then mixed into a basal media to produce the growth media 130. For example, a quantity of the growth factor can be mixed with a volume of a basal media including a quantity of salts, a quantity of sugars, a quantity of amino acids, and a volume of buffers to generate a volume of a growth media 130.

The genetically modified population of insects 100 can be configured to produce a set of growth factors, such as: basic fibroblast growth factor (or “FGF2”); transforming growth factor beta (or “TGF”); insulin-like growth factor (or “IGF”); transferrin; platelet-derived growth factor (or “PDGF”); vascular endothelial growth factor (or “VEGF”) angiopoietin; epidermal growth factor; colony-stimulating factors; tumor necrosis factor-alpha (or “TNFα”); etc. In one implementation, a singular genetically-modified population of insects 100 can be configured to produce multiple growth factors. Alternatively, in another implementation, the singular genetically-modified population of insects 100 can be configured to produce a particular growth factor. In this implementation, multiple populations of insects 100 can be propagated, each population of insects 100 genetically-modified to produce a particular growth factor, in a set of growth factors.

Once a growth factor is generated and extracted from a population of insects 100, an amount (e.g., quantity, proportion, concentration) of the growth factor can be included in a volume of the growth media 130 at a particular concentration according to a type of growth factor and/or a type of growth media 130. In one example, a volume of a growth media 130 including Basic Fibroblast Growth Factor (or “FGF2”) can include a quantity of FGF2 at a concentration of 0.1 milligrams per Liter. In another example, a volume of growth media 130 including Transforming Growth Factor Beta (or “TGF”) can be configured to include a quantity of TGF at a concentration of 0.002 milligrams per Liter. In another example, a volume of growth media 130 including Insulin-like Growth Factor (or “IGF”) can include a quantity of IGF at a concentration of 19.4 milligrams per Liter. In yet another example, a volume of growth media 130 including transferrin can include a quantity of transferrin at a concentration of 10.4 milligrams per Liter.

### 4. Drosophila

In one implementation, the population of insects 100 is a population of Drosophila genetically modified to produce a target compound 120.

As described above, “Drosophila” is referred to herein as a genus of flies belonging to the family Drosophilidae, members of which may be referred to as “small fruit flies,” “pomace flies,” “vinegar flies,” and/or “wine flies.” A population of Drosophila can include flies of a particular species, such as D. melanogaster (or “the common fruit fly”), D. immigrans, D. innubila, D. funebris, D. neotestacea, D. virilise, D. hydei, etc. For example, a population of D. melanogaster can be cultivated and configured for production of recombinant proteins. For example, the population of insects 100 can include a population of Drosophila melanogaster.

A genome of the population of Drosophila can be genetically-modified to include a target sequence 112 encoding for the target compound 120. Once the genome is genetically-modified and inserted into the population of Drosophila (e.g., as embryos), the population of Drosophila can grow, reproduce, and propagate the genome through each subsequent generation of the population. Further, during a growth period, the population of Drosophila can be fed a simple and inexpensive diet including cornmeal-based gelatinous foods. Therefore, the population of Drosophila including the genetically-modified genome can be relatively inexpensive to maintain. In addition, because Drosophila exhibit relatively short lifespans (e.g., less than 50 days), new generations can be rapidly produced, enabling frequent collection of the target compound 120 from the population of Drosophila. Therefore, a large quantity of the target compound 120 can be collected over a relatively short period of time.

Unlike cell-based systems, Drosophila include immune systems and therefore exhibit lower risk of contamination within the population of Drosophila. Further, unlike bacterial systems, Drosophila can implement post-translational modification to produce biologically active recombinant proteins (i.e., biologically active target compound 120S 120).

In one implementation, the genome of the population of Drosophila can be genetically-modified via P-elements, present in Drosophila, which are transposable elements that enable genes to move within the genome. These P-elements can cooperate with transposons to insert an exogenous gene inserted within a vector into the genome of the Drosophila population. These P-elements of Drosophila therefore enable insertion of an exogenous gene (i.e., the target sequence 112) and expression of a resulting protein (i.e., the target compound 120) encoded by the exogenous gene into the genome of the population of Drosophila. Alternatively, in another implementation, the genome of the population of Drosophila can be genetically-modified via a site-specific integrase system.

### 5. Genetic Modification of Insects

Block S102 of the method S100 recites genetically modifying a population of insects 100 to produce the first target compound 120. A population of insects 100 can be genetically modified to produce a particular target compound 120 (e.g., a particular growth factor). In particular, insect cells can be genetically modified to include a target sequence 112 encoding for the target compound 120, such that when the target sequence 112 is expressed, the insect cells generate the target compound 120.

In one implementation, the population of insects 100 can define a genetically-modified line of insects including multiple generations. In this implementation, a first generation of insects can be: genetically modified to produce a target compound 120 and then propagated to produce additional generations of insects configured to produce the target compound 120. For example, a first generation can be genetically modified to produce a target compound 120. In particular, a target sequence 112 encoding for the target compound 120 can be inserted into a genome of the first generation of insects. Once this target sequence 112 is stably integrated into the genome, this first generation can be propagated in cages over a propagation period, during which adult female insects in the first generation may lay eggs. These eggs develop into embryos which eventually hatch, thereby introducing new genetically-modified insect larvae defining a second generation of insects. The genome of this second generation thereby includes the target sequence 112—inherited from the first generation—encoding for the target compound 120. The target compound 120 can then be produced and collected from the second generation via the methods and techniques described below. Further, once the insect larvae in the second generation mature into adult insects, adult female insects in the second generation lay eggs and continue propagation of this line of genetically-modified insects. Therefore, in this implementation, once the genome of the first generation of insects is genetically modified to include the target sequence 112, this genome can be propagated through the line of insects, over multiple generations, without further genetic modification.

### 5.1. Regulatory Sequences

The genome of the population of insects 100 can be genetically modified to include a set of regulatory sequences upstream the target sequence 112. These regulatory sequences can be leveraged to control expression of the target sequence 112 and therefore control production of the target compound 120 expressed by the target sequence 112.

### 5.1.1 Promoter Sequence

In one implementation, the set of regulatory sequences can include a promoter sequence 114 coupled to the target sequence 112. The promoter sequence 114 can be associated with a particular stressor (e.g., heat-shock, cold-shock, nutrient-deprivation, dehydration, UV exposure), such that application of the particular stressor to the population of insects 100 activates expression of the promoter sequence 114. For example, the genome of the population of insects 100 can be genetically modified to include an HSP70 promoter sequence 114 upstream the target sequence 112, such that activation of the HSP70 promoter 114 leads to expression of the target sequence 112 and thereby generation of the target compound 120. The HSP70 promoter sequence 114 can activate in the presence of a heat-shock stressor in order to express heat-shock proteins which may protect cells from damage caused by the heat-shock stressor. More specifically, in response to a particular dosage of a heat-shock stressor, the HSP70 promoter sequence 114—bound by a set of transcription factors—can initiate transcription of sequences immediately downstream the HSP70 promoter sequence 114. Therefore, by coupling the target sequence 112 to the HSP70 promoter sequence 114 immediately upstream the target sequence 112, a heat-shock stressor can be implemented to control transcription of the target sequence 112.

Therefore, in the preceding example, a heat-shock stressor can be applied to the population of insects 100 to increase production of the target sequence 112 120. Thus, in this implementation, the genome of the population of insects 100 can be genetically modified to include: a target sequence 112 encoding for a particular target compound 120; and a promoter sequence 114 upstream the target sequence 112 and associated with a particular stressor. By coupling the target sequence 112 with a promoter sequence 114 upstream of the target sequence 112, expression of the promoter sequence 114 can be leveraged to control expression of the target sequence 112 and, therefore, to control generation of the target compound 120.

In another example, the population of insects 100 can be genetically modified to include a set of regulatory sequences including a promoter sequence 114 linked to a cold-shock stressor. In particular, the population of insects 100 101 can include: a promoter sequence 114 (e.g., coding for a heat-shock protein) associated with a stress response (e.g., cold stress response) of the population of insects 100 (e.g., Drosophila) to the cold-shock stressor, such that the promoter sequence 114 exhibits increased expression responsive to application of the cold-shock stressor; and a target sequence 112 120 linked (i.e., downstream) to the promoter sequence 114, such that expression of the promoter sequence 114 promotes expression of the target sequence 112 120. Therefore, in this example, the cold-shock stressor can be applied to the population of insects 100 to trigger increased expression of the promoter sequence 114 (e.g., coding for a particular heat-shock protein associated with a cold-stress response) and thereby trigger increased production of the target sequence 112 120.

In yet another example, the population of insects 100 can be genetically modified to include a set of regulatory sequences including a promoter sequence 114 linked to a UV stressor (i.e., an Ultraviolet radiation stressor or “UVR” stressor). In particular, in this example, the population of insects 100 101 can include: a promoter sequence 114 (e.g., coding for a particular heat-shock protein) associated with a stress response (e.g., a UV-stress response) of the population of insects 100 to the UV stressor, such that the promoter sequence 114 exhibits increased expression responsive to application of the UV stressor; and a target sequence 112 120 linked (i.e., downstream) to the promoter sequence 114, such that expression of the promoter sequence 114 promotes expression of the target sequence 112 120. Therefore, in this example, the UV stressor can be applied to the population of insects 100 to trigger increased expression of the promoter sequence 114 (e.g., coding for a particular heat-shock protein associated with a UV-stress response) and thereby trigger increased production of the target sequence 112 120. In yet another example, the population of insects 100 can be similarly genetically modified to include a set of regulatory sequences including a promoter sequence 114 (e.g., encoding for a particular heat-shock protein associated with a nutrient-stress response) linked to a nutrient stressor (e.g., a nutrient deficiency or other dietary stressor).

However, the population of insects 100 can be genetically modified to include promoter sequence 114s 114 (and/or additional regulatory sequences)—linked to downstream target sequence 112S 120—associated with other stressor types, such as environmental conditions, chemical agents, and/or biological agents.

### 6.1.1.1 Promoter Selection: Stressor-Specific

Generally, the genome of the population of insects 100 can be genetically-modified to include a promoter sequence 114 configured to regulate expression of the target sequence 112 downstream the promoter sequence 114.

In one implementation, the genome can be genetically-modified to include a promoter sequence 114—linked to the target sequence 112 corresponding to (e.g., encoding for) the target compound 120—configured to activate responsive to application of a particular stressor (e.g., a heat stressor, a chemical stressor, a nutrient stressor, a UV-light stressor) to the population of insects 100. Therefore, in order to regulate activation of the promoter sequence 114, the promoter sequence 114 can be selected based on a stressor type of the stressor applied during the treatment period for the population of insects 100, such that application of the stressor of the stressor type triggers activation of the promoter sequence 114 and thereby induces expression of the target sequence 112.

In one example, the genome can be genetically-modified to include a promoter sequence 114—such as an HSP70 promoter sequence 114, an HSP90 promoter sequence 114, an HSP60 promoter sequence 114, etc.—configured to activate responsive to application of a heat stressor (e.g., a heat-shock stressor, a cold-shock stressor). In another example, the genome can be genetically-modified to include a promoter sequence 114—such as a pTet promoter sequence 114—configured to activate responsive to application of a chemical stressor (e.g., tetracycline or a derivative thereof, a particular peptide). In yet another example, the genome can be genetically-modified to include a promoter sequence 114—such as an HSP70 promoter sequence 114, an HSP90 promoter sequence 114, an HSP60 promoter sequence 114, etc.—configured to activate responsive to application of a light stressor (e.g., a UV-light stressor, a pulsing-light stressor).

Therefore, Blocks of the method S100 can be executed to trigger production of a target compound 120 in a population of insects 100 responsive to application of any stressor (e.g., heat-shock, cold-shock, UV-light, chemical), based on the promoter sequence 114 included in the genetically-modified genome of the population of insects 100.

### 6.1.1.2 Promoter Selection: Compound-Specific

In another implementation, the promoter sequence 114 can be selected based on a set of target characteristics defined for the target compound 120. In particular, in this implementation, the population of insects 100 can be genetically-modified to produce a target compound 120 defining a set of target characteristics, such as a target structure and/or a target functionality. In this implementation, the genome of the population of insects 100 can be genetically-modified to include: a promoter sequence 114—linked to (e.g., upstream) the target sequence 112—configured to trigger transcription of the first target sequence 112 to trigger production of the first target compound 120 according to the set of target characteristics (e.g., the target structure and/or the target functionality); and the target sequence 112 linked to the promoter sequence 114 and corresponding to the target compound 120. The resulting target compound 120 can therefore be extracted from the population of insects 100—such as after application of the stressor and harvesting of the population of insects 100—and exhibit the set of target characteristics.

For example, a first population of insects 100 can be genetically-modified to produce a first target compound 120 defining a first target structure and a first target functionality. In this example, a first genome of the first population of insects 100 can be genetically-modified to include a first promoter sequence 114 associated with a first stressor (e.g., a heat-shock stressor) and configured to trigger transcription of a first target sequence 112—corresponding to the first target compound 120—to trigger production of the first target compound 120 according to the first target structure and the first target functionality. Then, during a first treatment period (e.g., succeeding a first growth period) for the first population of insects 100, one or more dosages of the first stressor can be applied to the first population of insects 100 to trigger production of the first target compound 120 exhibiting the first target structure and the first target functionality.

Further, in the preceding example, a second population of insects 100 can be genetically-modified to produce a second target compound 120 defining a second target structure and a second target functionality. In this example, a second genome of the second population of insects 100 can be genetically-modified to include a second promoter sequence 114 associated with a second stressor (e.g., a chemical stressor) and configured to trigger transcription of a second target sequence 112—corresponding to the second target compound 120—to trigger production of the second target compound 120 according to the second target structure and the second target functionality. Then, during a second treatment period (e.g., succeeding a second growth period) for the second population of insects 100, one or more dosages of the second stressor can be applied to the second population of insects 100 to trigger production of the second target compound 120 exhibiting the second target structure and the second target functionality.

By therefore including a different promoter sequence 114 in each of these genomes—and thus applying a different stressor during the corresponding treatment period—each target compound 120 can be configured to exhibit a target structure and functionality, such as corresponding to a final product or field (e.g., research, pharmaceutical, medical) associated with the final product. For example, while application of a heat stressor (e.g., heat-shock) during production of the first target compound 120 may yield the first target structure and the first target functionality, application of the heat stressor during production of the second target compound 120 may affect structure and/or functionality—such as by reducing protein stability and/or promoting protein aggregation—of the second target compound 120. Therefore, a different stressor—corresponding to the second promoter sequence 114—can be applied in order to trigger production of the second target compound 120.

### 6.1.1.3 Promoter Selection: Tissue-Specific

In yet another implementation, the promoter sequence 114 can be selected based on a tissue type of a target tissue selected for expression of the target compound 120. In particular, in this implementation, a genome of the population of insects 100 can be genetically-modified to include: a target sequence 112 corresponding to the target compound 120; a promoter sequence 114 linked to the target sequence 112 and configured to trigger expression of the target sequence 112 in tissue (e.g., cell tissue) of a particular tissue type. By regulating expression of the target sequence 112 within tissue of a particular tissue type, the promoter sequence 114—and/or additional regulatory elements, such as a set of tissue drivers, enhancers, leader sequences, etc.—can be configured to regulate: a rate of production of the target compound 120 in tissue of the tissue type; and/or characteristics (e.g., functionality, structure) of the resulting target compound 120.

For example, a first genome of a first population of insects 100 can be genetically-modified to include: a first promoter sequence 114—inducible by a first stressor—associated with cell tissue of a first tissue type; and a first target sequence 112 coupled to the first promoter sequence 114 and corresponding to a first target compound 120. Further, a second genome of a second population of insects 100 can be genetically-modified to include: a second promoter sequence 114—inducible by the first stressor—associated with cell tissue of a second tissue type; and the target sequence 112 coupled to the second promoter sequence 114 and corresponding to the first target compound 120. In this example, upon application of the first stressor to the first population of insects 100, the first target compound 120 can be produced at a first rate—in tissues of the first tissue type—in the first population of insects 100, and exhibit a first functionality, the first rate and the first functionality corresponding to the first tissue type. Upon application of the first stressor to the second population of insects 100, the first target compound 120 can be produced at a second rate—in tissues of the second tissue type—in the second population of insects 100, and exhibit a second functionality, the second rate and the second functionality corresponding to the second tissue type.

Therefore, the promoter sequence 114 can be configured to regulate production rate and characteristics—such as structure and/or functionality—of the target compound 120. Further, by producing a target compound 120 in a particular tissue or tissue type in the population of insects 100, the target compound 120 can be configured to exhibit characteristics mimicking characteristics of the target compound 120 when produced natively, such as in an animal (e.g., human, bovine).

### 6.1.1.4 Promoter Selection: Insect-Specific

In yet another implementation, the promoter sequence 114 can be selected based on an insect type of insects in the population of insects 100. In particular, the promoter sequence 114 can be selected based on compatibility with the insect type.

For example, a first genome of a population of Drosophila—modified to produce a first target compound 120—can be genetically-modified to include: a first promoter sequence 114 inducible by a first stressor; and a first target sequence 112 coupled to the first promoter sequence 114 and corresponding to the first target compound 120. In this example, a second genome of a population of mosquitos—modified to produce the first target compound 120—can be genetically-modified to include: a second promoter sequence 114 inducible by the first stressor or a second stressor distinct from the first stressor; and the target sequence 112 coupled to the second promoter sequence 114 and corresponding to the first target compound 120. In this example, the second genome can be configured to include the second promoter sequence 114—in replacement of the first promoter sequence 114—in order to enable integration of the second genome and/or production of the first target compound 120 within the population of mosquitos.

Additionally, in yet another implementation, the promoter sequence 114 can be selected based on a combination of the stressor type, the compound type, the tissue type, and/or the insect type.

### 5.1.2 Promoter Sequence+Leader Sequence

In one implementation, the set of regulatory sequences can include both the promoter sequence 114 and a leader sequence 116 upstream the target sequence 112. In this implementation, the leader sequence 116 can be configured to enable translation of the target sequence 112 during application of a particular stressor, while the promoter sequence 114 can be configured to enable transcription of the target sequence 112. In particular, the genome of the population of insects 100 can be genetically modified to include: a target sequence 112 encoding for a particular target compound 120; a leader sequence 116 upstream the target sequence 112; and a promoter sequence 114 upstream the leader sequence 116 and associated with a particular stressor. The leader sequence 116 can be configured to activate translation of the target sequence 112 during application of the particular stressor once transcribed thereby enabling production of the target compound 120.

For example, a genome of a population of Drosophila can be genetically modified to include: a target sequence 112 encoding for a particular target compound 120; a 5′UTR leader sequence 116 upstream the target sequence 112; and an HSP70 promoter sequence 114 upstream the leader sequence 116 and associated with a heat-shock stressor. During application of the heat-shock stressor to the population of Drosophila, transcription is highly repressed. However, transcription remains enabled for genes involved in the heat shock response, such as the HSP70 promoter sequence 114. Therefore, by including the HSP70 promoter sequence 114 in the genome of the population of Drosophila, transcription of the target sequence 112 can be enabled during application of the heat-shock stressor, while transcription of other genes in the genome are repressed.

Further, during application of the heat-shock stressor, translation of genes in the genome is also repressed. In this example, the 5′UTR leader sequence 116 downstream the HSP70 promoter sequence 114 is configured to enable translation at increased temperatures at which non-heat-shock mRNAs exhibit decreased translation. Therefore, by including the 5-UTR leader sequence 116, translation of the target sequence 112 can also be enabled during application of the heat-shock stressor. Thus, in this example, the HSP70 promoter sequence 114 and the 5′UTR leader sequence 116 cooperate to enable both transcription and translation of the target sequence 112 during application of the heat-shock stressor, while other genes in the genome exhibit repressed transcription and translation. The target compound 120 can therefore be produced by the population of Drosophila with increased enrichment (e.g., increased mass of the target compound 120 per unit mass of Drosophila flies).

Additionally and/or alternatively, in another implementation, the set of regulatory sequences can include both the promoter sequence 114 and an upstream activating sequence (or “UAS”) upstream the target sequence 112. In this implementation, the upstream activating sequence can be configured to activate transcription of the target sequence 112. For example, a genome of a population of Drosophila can be genetically modified to include: a target sequence 112 encoding for a particular target compound 120; an upstream activating sequence upstream the target sequence 112 and configured to bind with a transcription factor (e.g., Gal4 transcription factor) to activate transcription of the target sequence 112; and a HSP70 promoter sequence 114 upstream the target sequence 112 and associated with a heat-shock stressor. In this example, the genome can be genetically modified to include a particular sequence encoding for the transcription factor configured to bind with the activating sequence. Therefore, in this example, the upstream activating sequence and the transcription factor can cooperate to regulate transcription of the target sequence 112.

In another implementation, the genome of the population of insects 100 can be configured to include a test sequence 118 configured to confirm insertion of the target sequence 112 into the genome. For example, the genome can include a test sequence 118 corresponding to a “white+” gene in Drosophila which, when expressed, results in red-eyed Drosophila. The test sequence 118 can be coupled to the target sequence 112 and flanked by the P-Element insertion sites. Injection can occur in white-eyed Drosophila. Therefore, by coupling the test sequence 118 to the target sequence 112, insertion of the target sequence 112 and the associated regulatory sequences can be confirmed via visual confirmation of expression of the test sequence 118 (e.g., via visual confirmation of red-eyed Drosophila).

### 5.1.3 Secondary Elements

In one variation, the genome of the population of insects 100 can be configured to include a set of secondary elements configured to regulate transcription and translation of the target compound 120 within the population of insects 100.

In one example, the genome can be configured to include a set of tissue-specific drivers (e.g., Gal4, QF) configured to promote tissue-specific expression of the target compound 120 within the population of insects 100. Additionally and/or alternatively, in another example, the genome can be configured to include a set of regulatory elements configured to regulate levels of protein translation and/or mRNA stability, such as a set of ftz introns configured to stabilize mRNA and/or a set of gypsy insulators configured to limit position effects due to selection of the genomic location for insertion of the transgene.

However, the genome can be configured to include any other regulatory elements configured to regulate expression of the target compound 120 within the population of insects 100.

### 5.2 Multiple Target Sequences

In one variation, the population of insects 100 can be genetically modified to produce a set of target compound 120S 120. In particular, cells of the population of insects 100 can be genetically modified to include a set of target sequence 112S 112 corresponding to the set of target compound 120S 120. Therefore, in this variation, each insect in the population of insects 100 can be configured to generate multiple target compound 120S 120.

In one implementation, a population of insects 100 (e.g., Drosophila) can be genetically modified to produce a first target compound 120 and a second target compound 120 responsive to exposure to a first stressor (e.g., a heat stressor, a light stressor, a chemical stressor), a genome of the population of insects 100 including: a first promoter sequence 114 configured to activate responsive to exposure to the first stressor; a first target sequence 112 linked to the first promoter sequence 114 and corresponding to the first target compound 120; and a second target sequence 112 linked to the first promoter sequence 114 and corresponding to the second target compound 120. In this implementation, during a first treatment cycle, a first dosage of the first stressor can be applied to the population of insects 100 to: trigger production of the first target compound 120 and trigger production of the second target compound 120, such as at rates proportional to the first dosage. Additionally, in this implementation, during a second treatment cycle, a second dosage of the first stressor can be applied to the population of insects 100 to: trigger production of the first target compound 120 and trigger production of the second target compound 120, such as at rates proportional to the second dosage.

### 5.2.1 Multiple Target Sequences: Multiple Promoter Sequences

In one implementation, each target sequence 112, in the set of target sequence 112S 112, can be coupled to a promoter sequence 114, in a set of promoter sequence 114S 114. Further, each promoter sequence 114, in the set of promoter sequence 114S 114, can be associated with a particular stressor, in a set of stressors (e.g., heat-shock, nutrient deficiency, UV exposure, dehydration). By coupling each target sequence 112, in the set of target sequence 112S 112, to a distinct promoter sequence 114, in the set of promoter sequence 114S 114, expression of each target sequence 112 can be controlled via application of the set of stressors associated with the set of promoter sequence 114s 114.

For example, cells of a population of Drosophila can be genetically modified to include: a first target sequence 112 encoding for a first target compound 120 (e.g., FGF2); a first promoter sequence 114 linked to the first target sequence 112 and associated with a first stressor (e.g., heat-shock); a second target sequence 112 encoding for a second target compound 120 (e.g., IGF1); and a second promoter sequence 114 linked to the second target sequence 112 and associated with a second stressor (e.g., UV exposure). Once this genetically-modified population of Drosophila is grown to a particular stage (e.g., larvae, pupae, adult), the first and second stressor can be applied at particular dosages, such as at particular quantities and intervals, to control production of the first and second target compound 120S 120 within the population of Drosophila. In this example, the second target sequence 112 can be distinct or equivalent the first target sequence 112, such that generation of the first target compound 120 and the second target compound 120 can be controlled via application of the same stressor—such that the second stressor is equivalent the first stressor—or generation of the first target compound 120 and the second target compound 120 can be controlled via application of distinct first and second stressors, respectively.

Therefore, in this variation, a single genome of a population of insects 100 can be configured to generate multiple target compound 120S 120, generation of each target compound 120 controlled via application of various stressors to the population of insects 100. Thus, an entire set of growth factors required for production of a particular growth media 130—such as TGF-β, FGF2, IGF1, and transferrin for production of a growth media 130 for maintenance of stem cells in culture—can be generated and extracted from a single population of insects 100.

### 5.2.2 Multiple Target Compounds: Leader Sequence

In one implementation, the genome of the population of insects 100—genetically-modified to produce a first target compound 120 and a second target compound 120—can be genetically-modified to include: a first target sequence 112 corresponding to the first target compound 120; a second target sequence 112 corresponding to the second target compound 120; a leader sequence 116 (e.g., a spliced leader sequence 116)—coupled to the first and second target sequence 112S—configured to regulate translation of the first and second target sequence 112S; and a first promoter sequence 114 coupled to the leader sequence 116—and thereby coupled to the first and second target sequence 112S via the leader sequence 116—configured to activate responsive to exposure to a first stressor and configured to regulate transcription of the first and second target sequence 112S.

### 5.2 Detectable Signal Linked to Target Compound Generation

In one variation, the population of insects 100 is genetically modified to generate a detectable signal (e.g., an optical signal) indicating production of the target compound 120 by the population (e.g., when stressed). In particular, cells of the population of insects 100 can be genetically modified to include a reporter sequence linked to the promoter sequence 114 and/or target sequence 112 such that expression of the reporter sequence is linked to expression of the target sequence 112 in the population. Thus, expression of the reporter sequence by insects in the population can be configured to generate a signal (fluorescence, bioluminescence, and/or pigmentation change) that is directly detectable (e.g., with optical sensors or the human eye) and interpretable as an amount of the target compound 120 present in the insect population without necessitating harvesting and/or chemical testing of the insect population. More specifically, in this variation, the population of insects 100 are genetically modified to include a reporter sequence linked to the promoter sequence 114 and/or to target sequence 112 to enable direct tracking of composition of the target compound 120 within the insect population 100 before, during, and after exposure to a stressor—and prior to harvesting the insect population 100—based on expression of the reporter sequence (e.g., presence and/or magnitude of a signal generated by the reporter sequence) within the insect population 100.

For example, the genome of the population of insects 100 can be genetically modified to include: a target sequence 112 encoding for a particular target compound 120; a promoter sequence 114 coupled to the target sequence 112; and a reporter sequence coupled to the promoter sequence 114. The reporter sequence can be configured to encode for a fluorescent protein, such that when the reporter sequence is expressed, a detectable fluorescent signal is generated. Therefore, a user (e.g., a lab technician, a researcher) may monitor fluorescence of insects in the population of insects 100 to confirm production of the particular target compound 120.

Further, in this variation, the user may estimate or measure (e.g., via a spectrometer) a magnitude of the signal to estimate an amount (e.g., quantity, proportion, concentration) of the particular target compound 120 within the population of insects 100. In the preceding example, the user may record an intensity of fluorescence generated by the population of insects 100 via a handheld spectrometer. This intensity measurement can be leveraged to estimate an amount (e.g., quantity, proportion, concentration) of the particular target compound 120 within the population of insects 100, and the population of insects 100 can be continuously exposed or re-exposed to the stressor until the intensity of fluorescence of the insect population reaches an intensity of fluorescence corresponding to a target amount (e.g., exceeding a threshold amount) of the particular target compound 120.

Therefore, in this variation, by pairing the promoter sequence 114 with a reporter sequence configured to generate a detectable signal when expressed, production of the target compound 120 can be readily confirmed. Furthermore, in this variation, the population of insects 100 can be exposed to a stressor in order to produce the target compound 120 and the secondary signal, tested for magnitude of the secondary signal, and re-exposed to the stressor in order to increase magnitude of the secondary signal—and thus production of the target compound 120—up to a target signal magnitude that corresponds to a target proportion of the target compound 120 within the insect population.

In one variation in which the genome of the population of insects 100 is genetically-modified to include multiple unique target sequence 112S 112 configured to generate multiple unique target compound 120S 120, the genome can be further genetically-modified to include multiple reporter sequences 119. In this variation, the genome of the population of insects 100 can be genetically modified to include: a set of target sequence 112S 112 encoding for a set of target compound 120S 120; a set of promoter sequence 114S 114, each promoter sequence 114 coupled to a particular target sequence 112, in the set of target sequence 112S 112, and associated with a particular stressor, in a set of stressors; and a set of reporter sequences 119, each reporter sequence 119 coupled to a particular target sequence 112, in the set of target sequence 112S 112, and configured to express in response to expression of the particular target sequence 112.

### 5.4 Plasmid Generation and Insertion

In one implementation, the population of insects 100 can be genetically modified via insertion of a plasmid 110 (e.g., a pUAST plasmid, a pCaSpeR plasmid)—including the target sequence 112 encoding for the target compound 120—into the genome of the population of insects 100. The plasmid 110 can also be configured to include a set of regulatory sequences, such as the promoter sequence 114 and/or the leader sequence 116. In particular, the target sequence 112 and these regulatory sequences can be cloned via restriction enzyme cloning into a plasmid 110 including a multiple cloning site (or “MCS”). The target sequence 112 corresponding to the target compound 120 can be amplified via Polymerase Chain Reaction (or “PCR”) with the addition of restriction cut sites for a set (e.g., one or two) of the restriction enzymes added onto 5′ and 3′ amplification primers, thus enabling inserting of the target sequence 112 into the vector at the MCS.

The resulting plasmid no vector including the target sequence 112 (e.g., a recombinant protein-coding sequence) can then be transformed into chemically competent bacterial cells for propagation. In particular, the plasmid no vector can be transformed via ampicillin selection. Once the plasmid 110 vector is propagated in the bacterial culture, the plasmid 110 DNA can be extracted from the bacterial culture and purified. Successful insertion of the target sequence 112 into the plasmid no vector can be verified by sequencing upstream and downstream from the insertion site (i.e., the MCS).

Upon confirmation of insertion of the target sequence 112 into the plasmid 110 vector, the target sequence 112 can be inserted into the genome of the population of insects 100. Once the target sequence 112 is integrated into the insect genome, the population of insects 100 can be propagated for production of the target compound 120.

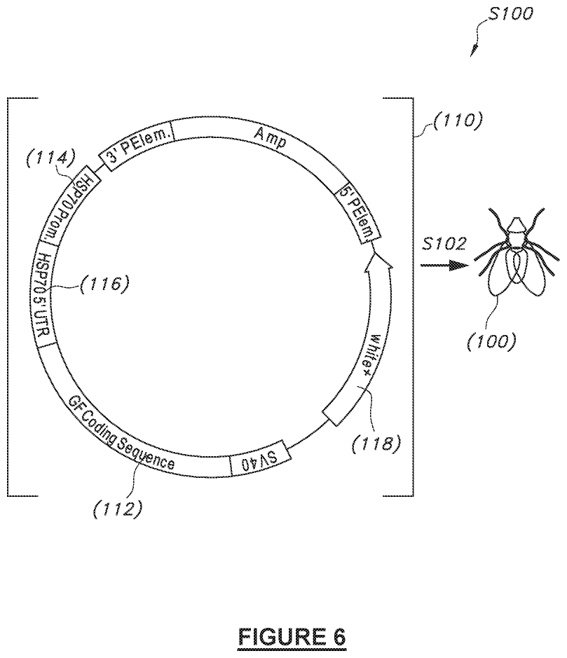

In one example, a population of Drosophila can be genetically modified to produce a target compound 120 via insertion of a recombinant pCaSpeR plasmid no into Drosophila embryos. In this example, as shown in FIG. 6, the plasmid no can be configured to include: a target sequence 112 encoding for a particular target compound 120; a set of regulatory sequences including a leader sequence 116 upstream the target sequence 112, and a promoter sequence 114 (e.g., an HSP70 promoter sequence 114) upstream of the leader sequence 116; and an ampicillin resistance gene configured to enable selection of transformants during propagation of the plasmid 110 in bacterial cells; a set of P-element sites configured for insertion of the plasmid 110 into Drosophila embryos; an MCS including a set of cut sites corresponding to a set of restriction enzymes (e.g., EcoRI, BgIII, Notl, Xhol, Kpnl, and Xba1); and a test sequence 118 (e.g., corresponding to the white+gene) configured to enable visual confirmation of propagation of the target sequence 112 in the population of Drosophila.

In this example, the target sequence 112 and regulatory sequences can be cloned via restriction enzyme cloning into the pCaSpeR plasmid 110. In particular, the target sequence 112 and regulatory sequences can be amplified via PCR with the addition of restriction cut sites for a subset (e.g., one or two) of restriction enzymes, in the set of restriction enzymes, added onto 5′ and 3′ amplification primers, thereby enabling insertion of the target sequence 112 into the pCaSpeR vector at the MCS via implementation of a T4 DNA ligase. Because the pCaSpeR vector includes the ampicillin resistance gene, the resulting pCaSpeR vector including the target sequence 112 can then be transformed into chemically competent bacterial cells for propagation via ampicillin selection. After propagation of the pCaSpeR vector in the bacterial culture, the plasmid 110 DNA can be extracted and purified. To confirm insertion of the target sequence 112 into the pCaSpeR vector, the plasmid 110 DNA can be sequenced upstream and downstream the MCS.

Once insertion of the target sequence 112 into the pCaSpeR vector is confirmed, the target sequence 112 can be inserted into the Drosophila genome via P-element insertion. A P-element transposon present in the plasmid 110 vector at a P-element site, in the set of P-element sites, can be leveraged to integrate the exogenous target sequence 112 into the insect genome in the presence of a transposase. In particular, the target sequence 112 can be integrated into the Drosophila genome via germline transformation including microinjection of the plasmid 110 DNA and a helper plasmid 110 including transposase, into a recipient Drosophila embryo. Therefore, in this example, the plasmid 110 DNA can be physically delivered to a posterior pole of the syncytial blastoderm at which precursors of the germ cells form. Upon cellularization of the embryo, the plasmid 110 DNA—including the set of P-element sites—can be integrated into the genome of the germ cells via interactions between the transposase and P-elements at the set of P-element sites.

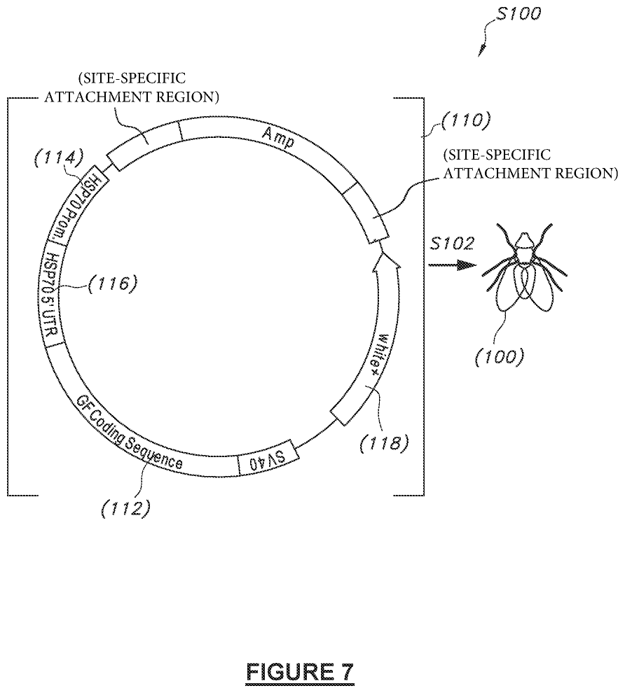

As described in the preceding example, in one implementation, as shown in FIG. 6, the population of insects 100 can therefore be genetically-modified to produce the target compound 120 via P-element based genome integration. However, the population of insects can be genetically-modified to produce the target compound 120 via any other known genetic engineering technique. For example, in one implementation, as shown in FIG. 7, the population of insects can be genetically-modified to produce the target compound 120 via implementation of a site-specific integrase system. In particular, in this implementation, in this implementation, the population of insects can be genetically modified to produce the target compound 120 via implementation of a recombinant pJFRC81 plasmid. Enzyme integrases can then be leveraged to integrate the exogenous target sequence 112 into the insect genome via recombination between attachment sites (e.g., attB and attP) on the vector backbone and Drosophila genomes.

### 5.4.1. Example: Generation of Genetically-Modified Population of Drosophila

In one example, a population of Drosophila can be genetically modified to produce a target compound 120. In this example, the target sequence 112 (i.e., coding sequence)—coding for the target compound 120 (e.g., a target recombinant protein)—is cloned via restriction enzyme cloning into a pUAST plasmid including a multiple cloning site (or “MCS”). The target sequence 112 is then amplified by PCR with the addition of restriction cut sites for one or two of the restriction enzymes added onto 5′ and 3′ amplification primers, thereby enabling insertion of the target sequence 112 into the pUAST plasmid vector at the MCS via a T₄ DNA ligase.

The resulting plasmid vector—including the target sequence 112 (e.g., the recombinant protein sequence)—can then be transformed into chemically competent bacterial cells for propagation. In this example, this transformation can be performed under ampicillin selection due to the presence of an ampicillin resistance gene in the pUAST vector. After the plasmid vector is thus propagated in the bacterial culture, the plasmid DNA is extracted and purified. Insertion of the target sequence 112 (e.g., the recombinant protein-coding sequence) into the pUAST vector can then be verified by sequencing upstream and downstream from the insertion site.

Upon confirmation of insertion into the plasmid (i.e., the pUAST plasmid vector), the target sequence 112 can be stably inserted into the Drosophila genome. For example, the target sequence 112 can be stably inserted into the Drosophila genome via a P-element transposon present in the plasmid vector. In particular, the pUAST vector includes P-element sites that—in the presence of a transposase—enable stable integration of exogenous DNA into the Drosophila genome. The target sequence 112 can be inserted via germline transformation through microinjection of the modified plasmid DNA (i.e., the modified pUAST plasmid vector) and a helper plasmid—including transposase—into a recipient Drosophila embryo. In this example, the plasmid DNA (e.g., the modified pUAST plasmid vector) is delivered to the posterior pole of the syncytial blastoderm, at which precursors of the germ cells are formed. Thus, upon cellularization of the embryo, the plasmid DNA (e.g., the modified pUAST plasmid vector)—including P-elements—is integrated into the genome of the germ cells via activity of the transposase.

### 5.5 Baseline Configurable Vector: Different Insect Types

In one implementation, the plasmid vector can be configured to integrate exogenous genes into insect populations of various types, such as Drosophila, Diptera, lepidoptera, Cicadida, etc.

In particular, in this implementation, a first instance of the plasmid vector can be configured for integration with a population of insects 100 of a first type (e.g., Drosophila) and a second instance of the plasmid vector can be configured for integration with a population of insects 100 of a second type (e.g., Diptera). Additional instances of the plasmid vector can be generated and integrated with additional populations of insects of various types.

For example, each instance of the plasmid vector can define a recognition site corresponding to the insect type. In particular, the recognition site for a particular plasmid vector can be selected based on the insect type, such that changing of this recognition site enables integration of the plasmid vector into genomes of various insect types. In one example, a first instance of the plasmid vector can define a first recognition site configured to enable integration of the first instance of the plasmid vector into a population of Drosophila. Additionally, in another example, a second instance of the plasmid vector can define a second recognition site configured to enable integration of the second instance of the plasmid vector into a population of Mediterranean fruit flies. Therefore, by modifying the recognition site of the plasmid vector, the plasmid vector can be configured for insertion in insects of different insect types.

Further, because many types of insects exhibit relatively similar genomes, the plasmid vector can be readily adapted—such as by modifying the recognition site as described above—to enable compatibility with a variety of insect types (e.g., genus, species).

Therefore, in order to maximize yield of a particular target compound 120, a population of insects 100 of a particular insect type—configured to produce the particular target compound 120 at a relatively high rate—can be selected for genetic-modification and production of the particular target compound 120. The plasmid vector can then be modified based on the selected insect type, such as by selecting a recognition site best matched to this insect type.

### 6. Growing the Population of Insects

Block S110 of the method S100 recites, during a growth period, cultivating the population of insects 100 according to a set of growth conditions. During the growth period, the population of insects 100 can be grown from a first stage (e.g., embryo) to a second stage (e.g., larvae, pupae, adult) over a duration of the growth period. During this growth period, the population of insects 100 can be subjected to a set of growth conditions (e.g., temperature, nutrient availability) configured to increase survival rates of insects in the population of insects 100 for the duration of the growth period.

In one implementation, the population of insects 100 can be cultivated during the growth period spanning a transition of the population of insects 100 from an embryonic stage to a larval stage.

For example, during the growth period, genetically-modified embryos of a population of Drosophila can be incubated at a set temperature of approximately (e.g., two degrees Celsius) twenty-five degrees Celsius. Once these embryos hatch to produce larvae, the population of Drosophila can be fed a particular diet (e.g., of cornmeal-based gelatinous foods). The population of Drosophila can be subjected to these conditions throughout the duration of the growth period, without introduction of a stressor.

In one implementation, the growth period can define a duration corresponding to transition to a particular life stage. For example, the population of Drosophila in the preceding example can be incubated at the set temperature over a first duration (e.g., 4 days) spanning a transition from embryo to a third instar larval stage. In this example, by terminating the growth period at the third instar larval stage, an amount (e.g., concentration, quantity) of the target compound 120 generated by the population of Drosophila can be quickly harvested before degradation of the target compound 120. Alternatively, in another example, the population of Drosophila can be incubated at the set temperature over a second duration (e.g., 5 days) spanning a transition from embryo, to larvae, to pupae. Alternatively, in yet another example, the population of Drosophila can be incubated at the set temperature over a third duration (e.g., 10 days, 15 days, 30 days) spanning a transition from embryo, to larvae, to pupae, to adult. In this example, the third duration can be selected to enable egg laying by female Drosophila prior to termination of the growth period for continued propagation of the target sequence 112 within the population of Drosophila.

### 7. Stressor Treatment

Block S120 of the method S100 recites, during a first treatment period succeeding the growth period, applying a first dosage of a first stressor to the population of insects 100, the first stressor configured to trigger production of the target compound 120 by the population of insects 100. After completion of the growth period, the population of insects 100 can be subjected to a stressor configured to control enrichment (e.g., unit mass of target compound 120 per unit mass of insects) of the target compound 120 within the population of insects 100. In particular, the first stressor can be configured to control enrichment of the target compound 120 via selective activation of the promoter sequence 114 associated with the first stressor and coupled to the target sequence 112.

For example, the genome of a population of Drosophila can be configured to include: a target sequence 112 encoding for a target compound 120; and a promoter sequence 114 associated with a heat-shock stressor and coupled to the target sequence 112. Then, during a first treatment period succeeding the growth period, the population of Drosophila can be subjected to a first dosage (e.g., an amount and duration) of the heat-shock stressor. In particular, in this example, the population of Drosophila can be shifted from incubation at the set of growth conditions (e.g., 25 degrees Celsius) to incubation at an increased temperature (e.g., between 35 degrees Celsius and 40 degrees Celsius). By subjecting the population of Drosophila to this heat-shock stressor, the promoter sequence 114 can be activated, thereby inducing expression of the target compound 120 encoded by the target sequence 112 coupled to the promoter sequence 114.

In one implementation, the treatment period can include a set of treatment cycles. In this implementation, the population of insects 100 can be subjected to multiple treatment cycles (e.g., 2 treatment cycles, 3 treatment cycles, 5 treatment cycles) during the treatment period in order to increase enrichment of the target compound 120 within the population of insects 100. For example, during a treatment period, a first dosage (e.g., a 12-degree temperature increase applied over 45 minutes) of a first stressor (e.g., heat-shock stressor) can be applied to the population of insects 100 over a first treatment cycle within the treatment period. Additionally, the first treatment cycle can also include a first rest period following application of the first dosage of the first stressor, during which the population of insects 100 can be returned to the set of growth conditions of the growth period. After this first rest period, a second dosage (e.g., approximating the first dosage, greater than the first dosage) can be applied to the population of insects 100 over a second treatment cycle within the treatment period. Similarly, the second treatment cycle can include a second rest period. Additionally, the treatment period can include a third treatment cycle, a fourth treatment cycle, and so on, in order to increase enrichment of the target compound 120. Therefore, the treatment period can include a particular quantity of treatment cycles configured to maximize enrichment of the target compound 120 within the population of insects 100.

### 7.1 Stressor Tolerance

In one implementation, Blocks of the method S100 recite: during a first treatment cycle within the treatment period, applying a first dosage of a first stressor to the population of insects 100; and, during a second treatment cycle succeeding the first treatment cycle within the treatment period, applying a second dosage of the first stressor, the second dosage greater than the first dosage, to the population of insects 100 to trigger production of the target compound 120.

In this implementation, the insect population can be exposed to stressor doses of increasing magnitude over successive treatment cycles in order to build tolerance to the stressor within the population of insects 100. For example, during a growth period, a population of insects 100 can be cultivated according to a first set of growth conditions defining a first temperature range—such as within a threshold deviation of 25 degrees Celsius—for the growth period. Then, during a first treatment cycle, within a treatment period succeeding the growth period, the population of insects 100 can be located within a treatment chamber and held at temperatures—within the treatment chamber—within a second temperature range (e.g., between 30 degrees Celsius and 35 degrees Celsius, between 35 degrees Celsius and 37 degrees Celsius), temperatures within the second temperature range exceeding temperatures within the first temperature range. Then, during a second treatment cycle, succeeding the first treatment cycle within the treatment period, the population of insects 100 can be located (or maintained) within the treatment chamber and held at temperatures within a third temperature range (e.g., between 35 degrees Celsius and 37 degrees Celsius, between 37 degrees Celsius and 40 degrees Celsius), temperatures within the third temperature range exceeding temperatures within the second temperature range.

Additionally and/or alternatively, in another implementation, a rate of production and/or an amount of the target compound 120 produced can be controlled via the dosage of the stressor applied to the population of insects 100. However, if the dosage of the stressor is increased too quickly, the population of insects 100 may not survive application of the stressor. Therefore, a lower dosage of the stressor can initially be applied during a first treatment cycle, followed by a higher dosage during a subsequent treatment cycle, thus increasing tolerance of the insects to the stressor and enabling application of increased dosages of the stressor for increased enrichment of the target compound 120 within the population of insects 100.

In particular, in this implementation, during a treatment period: during a first treatment cycle within the treatment period, a first dosage of a first stressor can be applied to the population of Drosophila to trigger production of the first target compound 120 in the population of Drosophila at a first rate proportional the first dosage; and, during a second treatment cycle within the treatment period, a second dosage, exceeding the first dosage, of the first stressor can be applied to the population of Drosophila to trigger production of the first target compound 120 in the population of Drosophila at a second rate proportional the second dosage and exceeding the first rate.

For example, on Day-20 of a lifespan of a population of Drosophila, a first dosage (e.g., a five-degree temperature increase applied over 15 minutes) of a first stressor (e.g., a heat-shock stressor) can be applied to the population of Drosophila over a first treatment cycle. During the first treatment cycle, the first dosage of the first stressor can be configured to trigger production of the target compound 120 at a first rate. Five days later, on Day-30 of the lifespan, a second dosage (e.g., an eight-degree temperature increase applied over 30 minutes) of the first stressor can be applied to the population of Drosophila over a second treatment cycle, the second dosage greater than the first dosage. During the second treatment cycle, the second dosage of the first stressor can be configured to trigger production of the target compound 120 at a second rate greater than the first rate. Finally, two days later, on Day-32 of the lifespan, a third dosage (e.g., a twelve-degree temperature increase applied over a 45-minute treatment interval) can be applied to the population of Drosophila over a third treatment cycle, the third dosage greater than the first and second dosage. Similarly, the third dosage of the first stressor can be configured to trigger production of the target compound 120 at a third rate greater than the first and second rate.

### 7.2 Multiple Stressors

In one variation, the population of insects 100 can be configured to produce multiple target compound 120S 120. In this variation, each target compound 120 can be linked to a particular stressor, in a set of stressors, such that production of each target compound 120 can be regulated via application of the set of stressors.

For example, the population of insects 100 can be genetically modified to include: a first target sequence 112 encoding for a first target compound 120; a first promoter sequence 114 coupled to the first target sequence 112 and associated with a heat-shock stressor; a second target sequence 112 encoding for a second target compound 120; and a second promoter sequence 114 coupled to the second target sequence 112 and associated with a cold-shock stressor. In this example, the population of insects 100 can be exposed to both the heat-shock stressor and the cold-shock stressor in controlled proportions to produce both the first and second target compound 120S 120 in controlled proportions. Therefore, a single population of insects 100 can be configured to produce two different target compound 120S 120, thereby increasing redundancy across multiple different insect populations 100 with different combinations of promoter sequence 114s 114 and target sequence 112S 112.

In this variation, each stressor, associated with a promoter sequence 114 112, can be applied to the population of insects 100 over multiple treatment cycles to increase enrichment of the target compound 120S 120 within the population of insects 100. In particular, in the preceding example, the population of insects 100 can be grown in a primary incubator set at 25 degrees Celsius during a growth period. After completion of the growth period, the population of insects 100 can be placed in an incubator at 30 degrees Celsius for a first duration of 10 minutes for a first cycle of the heat-shock stressor. Upon expiration of the first duration, the population of insects 100 can be placed back in the primary incubator at 25 degrees Celsius. After a first rest period (e.g., 15 minutes, 1 hour, 12 hours), the population of insects 100 can be placed in an incubator at 20 degrees Celsius for a second duration of 10 minutes for a first cycle of the cold-shock stressor. Upon expiration of the second duration, the population of insects 100 can again be placed in the primary incubator at 25 degrees Celsius. The first cycle of both the heat-shock and cold-shock stressors can be configured to increase tolerance of the population of insects 100 to these stressors before increasing magnitude of these stressors.

Later, after a second rest period (e.g., 15 minutes, 1 hour, 12 hours)—during which the population of insects 100 remain in the primary incubator at 25 degrees Celsius—the population of insects 100 can be moved into an incubator set at 37 degrees Celsius for a third duration of 45 minutes for a second cycle of the heat-shock stressor. Upon expiration of the second duration, the population of insects 100 can be placed in the primary incubator. After a third rest period, the population of insects 100 can be placed in an incubator at 15 degrees Celsius for a fourth duration of 45 minutes. Upon expiration of the fourth duration, the population of insects 100 can be placed in the primary incubator. A dosage (e.g., magnitude and duration) of both the first and second stressor can be increased and/or maintained over multiple treatment cycles to acclimate the population of insects 100 to these stressors and to increase enrichment of the first and second target compound 120S 120 within the population of insects 100.

### 8. Harvesting Insects

Block S130 of the method S100 recites, in response to a proportion of the first target compound 120 within the population of insects 100 exceeding a threshold proportion, harvesting the population of insects 100. After completion of the stressor treatment, the population of insects 100 can be harvested for collection of the target compound 120. In one implementation, the population of insects 100 can be harvested during a larval stage for insects in the population of insects 100. For example, the population of insects 100, in the larval stage, can be collected from a cage or chamber containing the population of insects 100 in preparation for homogenization.

Block S140 of the method S100 recites homogenizing the population of insects 100 to form a blend including the proportion of the first target compound 120 and a proportion of a set of secondary components. In one implementation, the population of insects 100 can be mechanically homogenized in a particular buffer matched to the target compound 120 to form a blend. For example, the euthanized population of insects 100 can be mixed in a blender with a particular buffer over a set duration to form a homogenous mixture (i.e. the blend) from which the target compound 120 can be readily extracted.

### 8.1 Target Amount of the Target Compound

In one implementation, the population of insects 100 can be harvested once a particular amount (e.g., exceeding a defined threshold amount) of the target compound 120 has been produced.

In particular, in this implementation, Blocks of the method S100 can include: predicting a first amount (e.g., concentration, quantity, proportion) of the first target compound 120 produced within the population of Drosophila during the treatment period; and, in response to the first amount exceeding a threshold amount defined for the treatment period, harvesting the population of Drosophila. For example, the population of insects 100 can be harvested in response to an amount (e.g., quantity, proportion, concentration) of the target compound 120 produced within the population of insects 100 exceeding a threshold amount, such as a threshold concentration of one percent, two percent, five percent, fifteen percent, thirty percent, etc.

In this implementation, in response to the amount of the first target compound 120 falling below the threshold amount, a subsequent stressor cycle—including application of the stressor to the population of insects 100—can be implemented in order to trigger further production of the first target compound 120 in the population of Drosophila.

Additionally, in a similar implementation—in which the population of insects 100 is genetically-modified to produce more than one target compound 120—the population of insects 100 can be harvested once a particular amount (e.g., exceeding a defined threshold amount for each of the target compound 120S) of the first and second target compound 120S 120 have been produced. For example, upon completion of a treatment cycle for a population of insects 100 configured to produce a first and second target compound 120, a first amount of the first target compound 120 and a second amount of the second target compound 120 can be predicted. In this example, in response to the first amount exceeding a first threshold amount defined for the first target compound 120 and, in response to the second amount exceeding a second threshold amount defined for the second target compound 120, the population of insects 100 can be harvested for extraction of the first and second compound.

### 8.1.1 Predicted Amount of Target Compound: Signal Detection

In one implementation, the amount of the target compound 120 produced by the population of insects 100—upon completion of one or more treatment cycles—can be predicted based on detection of a signal generated by the population of insects 100.

In particular, in this implementation, the genome of the population of insects 100 can include the reporter sequence 119 configured to generate a detectable signal (e.g., an optical signal) indicating generation of the target compound 120. In response to detecting this signal expressed by the reporter sequence 119, a magnitude of the detectable signal (e.g., an intensity of a fluorescence signal) can be interpreted. An amount of the target compound 120 produced within the population of insects 100 can then be estimated based on the magnitude of this signal. Alternatively, other methods and techniques can be implemented to estimate the amount (e.g., quantity, concentration, proportion) of the target compound 120.

In the preceding example, a known correlation (e.g., a historical and/or experimental correlation) between amounts of the target compound 120 and magnitudes of the signal can be leveraged to estimate the (current) amount of the target compound 120 present in the population of insects 100. Then, in response to harvesting this population of insects 100, the system can rectify this correlation based on an actual, measured amount of the target compound 120 100 present in the population of insects 100.

### 8.2 Predicted Amount of Target Compound: Target Quantity of Treatment Cycles

In another implementation, the amount of the target compound 120 produced by the population of insects 100—upon completion of one or more treatment cycles—can be predicted based on a quantity of treatment cycles completed and/or based on a dosage of the stressor applied during each treatment cycle.

In particular, in this implementation, the population of insects 100 can be harvested after a target quantity of treatment cycles configured to generate an amount (e.g., concentration, quantity, proportion) of the target compound 120 exceeding a threshold amount. In this implementation, the amount of the target compound 120 generated each treatment cycle can be predicted based on historical data collected for the target compound 120 during preceding treatment cycles. Over time, a target quantity of treatment cycles—and/or a target dosage of the stressor—configured to yield a target amount or threshold amount (e.g., minimum amount) of the target compound 120, can be identified, such as for a particular target compound 120, for a particular insect type, for a particular stressor, etc.

For example, for a particular target compound 120, for a first generation of the population of insects 100, a first quantity of treatment cycles can be applied. Upon extraction of the particular target compound 120 for this first generation, a first amount of the particular target compound 120 (e.g., mass of the particular target compound 120 per mass of the first generation of insects) can be measured and recorded. Later, for a second generation of the population of insects 100, a second quantity of treatment cycles can be applied. Upon extraction of the particular target compound 120 for this second generation, a second amount of the particular target compound 120 20 can be measured and recorded. Over time, this process can be repeated over numerous generations of the population of insects 100 to develop a model (e.g., linking quantity of treatment cycles to amounts of the target compound 120) for the particular target compound 120 for maximizing amounts of the particular target compound 120 within the population of insects 100. Therefore, based on the model, a set quantity of treatment cycles—configured to yield a maximum amount of the particular target compound 120—can be identified.

### 9. Protein Purification

Block S150 of the method S100 recites separating the proportion of the first target compound 120 from the second proportion of the set of secondary components for extraction of the proportion of the target compound 120 from the blend.

In one implementation, the target compound 120 can be extracted from the blend via affinity chromatography. In this implementation, the blend can be centrifuged to separate out any insoluble materials remaining from the insects, resulting in a supernatant (e.g., a homogenous mixture) including the proportion of the target compound 120 and the second proportion of the set of secondary components. This supernatant can be passed over a column including a stationary phase configured to capture the proportion of the target compound 120. For example, the stationary phase can include a particular type of beads (e.g., Sepharose beads) including antibodies or receptors configured to selectively bind to the target compound 120 to extract the proportion of the target compound 120 from the supernatant. In this implementation, the proportion of the secondary components of the supernatant can then be washed off of the column, such that only the proportion of the target compound 120 remains bound to the column. This proportion of the target compound 120 can then be eluted off of the beads of the column, such as by washing the column with a competitive ligand or a high salt solution configured to disrupt interactions between the target compound 120 and antibodies or receptors of the beads.

In another implementation, the target compound 120 can be extracted from the blend via size exclusion chromatography. In this implementation, the blend (e.g., a homogenous mixture) can similarly be centrifuged to separate out any solid materials, resulting in the supernatant (e.g., a homogenous mixture) including the proportion of the target compound 120 and the second proportion of the set of secondary components. The supernatant can then be run over a column including pores of a particular size range configured to trap biomolecules in the supernatant based on molecular size. For example, the column can be configured to include pores within the particular size range such that: smaller biomolecules in the supernatant become trapped in these pores and thus elute more slowly; and large biomolecule are blocked from entering these pores and thus elute more quickly. Therefore, retention times of the target compound 120 and each secondary component, in the set of secondary components, can be estimated based on molecular size. Thus, the target compound 120 can be separated and collected based on retention time within the column.

Additionally or alternatively, in another implementation, both affinity chromatography and size exclusion chromatography can be implemented in combination for extraction of the target compound 120 from the blend. For example, the supernatant can be run over a first column configured to purify the target compound 120 via size exclusion chromatography. Then, the supernatant can be run over a second column configured to further purify the target compound 120 via affinity chromatography. Alternatively, in yet another implementation, ion-exchange chromatography can be implemented for extraction of the target compound 120.

In another implementation, the target compound 120 can be extracted from the blend via three phase partitioning (or “TPP”). In this implementation, the proportion of the target compound 120 can be precipitated out of the supernatant via addition of t-butanol and ammonium sulfate. The t-butanol can bind to the proportion of the target compound 120 in the supernatant, enabling the proportion of the target compound 120 to float above the aqueous supernatant solution. The proportion of the target compound 120—floating above the aqueous solution—can then be extracted. This proportion of the target compound 120 can then be dissolved in a separate solution to regain functionality.

### 10. Protein Storage

The resulting target compound 120 can be further purified, processed, and/or stored according to a set of storage conditions defined for the target compound 120. In particular, the target compound 120 can define a set of storage conditions configured to: maximize a shelf-life of the target compound 120; and maintain and/or promote a set of target characteristics—such as the target structure and/or functionality—of the target compound 120.

In one implementation, the purified target compound 120 can be mixed with a volume of a buffer solution—matched to the target compound 120—to generate a first mixture. The first mixture—including the purified target compound 120—can then by lyophilized (or “freeze-dried”) to generate a lyophilized mixture in preparation for storage. This lyophilized mixture can then be stored according to a set of storage conditions defined for the target compound 120, such as at temperatures within a target temperature range configured to maintain structure and functionality of the target compound 120 for a duration exceeding a threshold duration.

Additionally, in this implementation, a set of stabilizers (e.g., stabilizing ingredients) can be added to the first mixture and/or to the lyophilized mixture in order to prolong stability of the target compound 120. For example, various proteins or sugars can be added to the first mixture and/or to the lyophilized mixture.

In one example: a first population of insects 100 can be configured to produce a first target compound 120 exhibiting a first functionality; and a second population of insects 100 can be configured to produce a second target compound 120 exhibiting a second functionality. In this example, a first amount of the first target compound 120—exhibiting the first functionality—can be: extracted from the first population of insects 100; mixed with a first set of stabilizing agents configured to stabilize the first target compound 120 exhibiting the first functionality; and stored under a first set of storage conditions configured to maintain the first functionality of the first target compound 120. Further, a second amount of the first target compound 120—exhibiting the second functionality—can be: extracted from the second population of insects 100; mixed with a second set of stabilizing agents configured to stabilize the first target compound 120 exhibiting the second functionality; and stored under a second set of storage conditions configured to maintain the second functionality of the first target compound 120.

### 11. Variation: Tissue-Specific Expression

In one implementation, the population of insects 100 can be genetically-modified to produce the target compound 120 in tissue of a particular tissue type.

In particular, in this implementation, the population of insects 100 can be genetically-modified to produce a first target compound 120 responsive to exposure to a first stressor, a genome of the population of insects 100 including: a first promoter sequence 114 configured to activate responsive to exposure to the first stressor and associated with a first tissue type in a set of tissue types; and a first target sequence 112 linked to the first promoter sequence 114 and corresponding to the first target compound 120. In this implementation, a dosage of the first stressor can be applied to the population of insects 100 to trigger production of the first target compound 120—in the population of insects 100—in tissues of the first tissue type, such as at a rate proportional the dosage of the first stressor. Additionally dosage of the first stressor can be applied to further trigger production of the first target product in tissues of the first tissue type during subsequent treatment cycles.

In this implementation, by enabling tissue-specific expression of the target sequence 112, the target compound 120 can be configured to exhibit characteristics linked to specific tissue types. For example, a first target compound 120 produced by a first population of insects 100—genetically-modified to produce the first target compound 120 in tissues of a first tissue type—can exhibit a first functionality and/or a first structure based on the first tissue type. Additionally, the first target compound 120 produced by a second population of insects 100—genetically-modified to produce the first target compound 120 in tissues of a second tissue type—can exhibit a second functionality and/or a second structure based on the second tissue type. Therefore, characteristics of the target compound 120 can be achieved by regulating expression of the target sequence 112 within tissues of a particular tissue type.

Further, the target compound 120 can be produced at a particular rate corresponding to the tissue type. For example, a first population of insects 100 can be configured to produce a first target compound 120 in tissues of a first tissue type at a first production rate. In this example, a second population of insects 100 can be configured to produce the first target compound 120 in tissues of a second tissue type—distinct from the first tissue type—at a second production rate less than the first production rate. Therefore, by selectively regulating expression of the target sequence 112—encoding for the first target compound 120—within tissues of the first tissue type, the first population of insects 100 can be configured to produce the first target compound 120 at a relatively higher rate than the second population of insects 100.

### 12. Other Target Proteins

As described above, the target compound 120 can define a growth factor configured for integration in a growth media. In one implementation, the target compound 120 can define a growth factor configured for integration in a growth media for culturing myoblast and myocyte cell lines. Alternatively, in another implementation, the target compound 120 can define a growth factor configured for integration in a growth media for cell culture in pharmaceutical and academic research applications.

In one variation, the target compound 120 can define a recombinant protein for integration in other applications such as: pharmaceuticals; therapeutics; vaccines; diagnostics; cosmetics; etc. For example, the target compound 120 can define a Cholera toxin B protein or a collagen protein.

The target compound 120 can be produced at different purities (or “grades”) based on the final product of the target compound 120. For example, the target compound 120 can be produced in a solubilized concentrate form for integration in a growth media for culturing cells. Alternatively, in another example, the target compound 120 can be produced at a pharmaceutical grade for production of growth media for cell culture in pharmaceutical and academic research application. This target compound 120 can be produced at a higher purity in order to achieve the pharmaceutical grade. Therefore, in this example, the population of insects 100 can be treated and/or maintained under a particular set of conditions in order to minimize contamination and increase purity of the final target compound 120.

### 13. Health Applications

In one implementation, the target compound 120 can be configured for integration in various therapeutic applications. For example, the target compound 120 can be configured for integration in various health applications, such as: pharmaceuticals, therapeutics, vaccines, diagnostics, etc.

In this implementation, the target compound 120 can be configured to exhibit a target functionality, such that the target compound 120 is both expressed—in sufficient quantities—and exhibits activity mimicking and/or equivalent activity of a corresponding native compound (e.g., a human counterpart protein). For example, a fibroblast growth factor protein—generated by the population of insects 100 according to the methods and techniques described above—can exhibit a target functionality corresponding to functionality of a human fibroblast growth factor protein (e.g., when expressed in humans), such that the fibroblast growth factor protein functions equivalent as the human fibroblast growth factor protein when introduced into human systems.

Further, the target compound 120 can be configured to exhibit compatibility with a particular animal system (e.g., human system), in order to mitigate against a native antibody response upon introduction of the target compound 120 into the particular animal system. For example, a prolactin protein generated by the population of insects 100 can be configured to exhibit structures and functionality similar and/or equivalent a human prolactin protein (e.g., when expressed in humans), in order to mask this “foreign” prolactin protein as a “native” human prolactin protein, and therefore mitigate against an undesirable antibody response when introduced into a human system.

In this implementation, the target compound 120 can further be configured to exhibit a target purity corresponding to a final product or application for the target compound 120. In particular, the target compound 120 can be purified—such as via column filtration for removal of secondary components and/or endotoxins generated in production of the target compound 120—prior to mixing with a buffer or additional stabilizing components for storage. These buffers and stabilizing components (e.g., sugars) can similarly be configured to exhibit a target purity, in order to minimize contamination and/or decreased purity of the target compound 120.

### 13.1.1 Human Systems: Post-Translational Modifications

In one implementation, the population of insects 100 can be configured to produce biologically-active recombinant protein (i.e., the target compound 120). More specifically, because insect systems exhibit capability of post-translational modifications, the population of insects 100 can produce the target compound 120—exhibiting a target functionality—due to implementation of these post-translational modifications (or “PTMs”).

In particular, PTMs—such as glycosylation, phosphorylation, ubiquitination, methylation, and acetylation—are chemical modifications that occur after generation of the protein and that are covalently linked to proteins. PTMs promote increased stability and functionality of the resulting proteins, and are involved in numerous cellular processes, such as cell division, growth, differentiation, signaling and regulation. While insect systems exhibit PTM structures, these insect PTM structures generally exhibit less complexity than mammalian PTM structures. Therefore, in various health-related applications—such as therapeutics and/or vaccines—in order to enable combability of the target compound 120 in mammalian systems (e.g., human systems), the population of insects 100 can be genetically-modified to produce insect PTM structures—included in the target compound 120—more-closely resembling and/or equivalent the mammalian PTM structures (e.g., human PTM structures). Therefore, by genetically-modifying the population of insects 100 to produce insect PTM structures—within the target compound 120—that correspond to the mammalian counterpart PTM structures, the target compound 120 can be configured to exhibit: enhanced binding to a target receptor protein (e.g., upon application in the mammalian system) and therefore increased stability (e.g., protein stability); increased target reactivity and/or functionality within the mammalian system; and minimal or no undesired reactivity.

### 13.1.2 Example: Glycosylation

In one example, the population of insects 100 can be configured to enable glycosylation—which may affect protein folding, conformation, stability, distribution, and activity—of the target compound 120.

Glycosylation is the addition of sugar moieties (mono, oligo, and polysaccharides) to protein molecules at specific amino acids. For example, N-glycosylation includes attachment of sugars to the nitrogen atom of the amino acid residue asparagine. In another example, O-glycosylation includes attachment of sugars to the oxygen atom of a serine or threonine residue. Dependent on the amino acid sequence and a structure of glycosylated proteins (or “glycoproteins”), glycosylated proteins can be embedded in the cell membrane, secreted through granules via exocytosis, or stored in lysosomes (e.g., for future use).

While human cells generate complex patterns of glycosylation, most available recombinant protein production platforms generate simpler glycosylation patterns. Further, while insect cells can generate simple N-glycan structures (oligomannosidic and paucimannosidic), insect cells generally do not generate N-glycan structures exhibit complexity equivalent mammalian N-glycan structures. However, insect cells do include a set of enzymes (e.g., glycosyltransferases) configured to produce hybrid and complex type N-glycan modifications. In particular, insect cells produce the simple type N-glycan structures due to removal of complex structures during activation of beta-N-acetylglucosaminidase (hereinafter “GlcNAcase”), which removes glucosamine moieties (GlcNAc) that generally facilitate generation of glycan structures exhibiting increased complexity

Therefore, in one implementation, the population of insects 100 can be genetically-modified to produce a target compound 120 exhibiting complex type N-glycans, via genetically-modifying the population of insects 100 to enable glycosylation and suppressing native insect cell activity that triggers removal of complex N-glycan structures. For example, the population of insects 100 can be genetically-modified to: suppress GlcNAcase activity via RNAi (transient and transgenic) and/or chemical inhibitors; and/or suppress activity of GlcNAcase and enable expression of of beta-1,4-galatosyltransferase (or “GalT”), an enzyme that promotes addition of sugar moieties—(e.g., galactose) onto existing simple type N-glycans.

### 13.1.3 Multiple Stocks

In one implementation: a first population of insects 100 can be genetically modified to produce a target compound 120 exhibiting a first set of PTMs corresponding to a first field of use; and a second population of insects 100 can be genetically modified to produce the target compound 120 exhibiting a second set of PTMs corresponding to a second field of use. Generally, in this implementation, depending on the field of use of the target compound 120, the population of insects 100 can be genetically modified to produce the target compound 120 exhibiting a particular set of PTMs required for this field of use.

For example, a first population of Drosophila can be modified to produce a fibroblast growth factor (or “FGF”) for integration in a human system. In this example, the first population of Drosophila can be modified to enable a first set of PTMs on the fibroblast growth factor, such that the resulting “humanized” fibroblast growth factor exhibits characteristics (e.g., structure, functionality) corresponding to an equivalent human counterpart fibroblast growth factor. In addition, in this example, a second population of Drosophila can be modified to produce a fibroblast growth factor (or “FGF”) for integration in a food system. In this example, the second population of Drosophila can be modified to produce the fibroblast growth factor with or without enabling PTMs on the resulting fibroblast growth factor. Therefore, this second population of Drosophila can be leverage to produce fibroblast growth factor for non-human system applications.

In one example, the method S100 can be executed to generate an FBS-alternative (e.g., a “synthetic” fetal bovine serum) including nutrients and a set of growth factors included in standard FBS derived from cow fetuses. In particular, in this example, the method S100 can be executed to produce a set of target compound 120S—corresponding to standard FBS—such as: Albumin; Fibroblast Growth Factor-2 (or “FGF2”); and Platelet-derived Growth Factor (or “PDGF”). The entomological lysate of Albumin (or “EntoA”) can be supplemented with amounts of FGF2 and PDGF—each derived from the population of insects 100—to generate an alternative FBS mimicking the standard FBS (e.g., derived from cow fetuses). Further, in this example, each target compound 120, in the set of target compound 120S, can be configured to exhibit a particular functionality and/or structure matched to a final product associated with the alternative FBS. For example, the set of target compound 120S can be configured to exhibit functionality and structures corresponding to human systems, such that the final product corresponds to an FBS alternative for implementation in human cell systems. In another example, the set of target compound 120S can be configured to exhibit functionality and structures corresponding to bovine systems, such that the final product corresponds to an FBS alternative for implementation in nonhuman cell systems.

### 13.2 Shelf-Life+Storage

In one implementation, to further extend a shelf-life and preserve functionality of the target compound 120, a cryoprotectant can be added to the resulting target compound 120, such as before or after lyophilization of the target compound 120.

For example, a first amount of a target compound 120 can be extracted from a blend including secondary components. This first amount of the target compound 120 can then be mixed with a volume of a buffer solution to generate a first mixture. Further, a cryoprotectant—such as a mixture of proteins (e.g., BSA, HAS) and/or sugars (e.g., Lacital, Mannital)—can be added to the first mixture to further prolong the stability and shelf-life of the target compound 120. Finally, the first mixture can be lyophilized to generate a lyophilized mixture, which can then be stored under a set of storage conditions selected for the target compound 120.

### 13.3 Example: Fibroblast Growth Factor (Human)

In one implementation, a population of insects 100 can be genetically modified to generate a fibroblast growth factor. Generally, in this implementation, Blocks of the method S100 can be executed to: cultivate a genetically-modified population of insects 100 configured to generate an fibroblast growth protein (i.e., the target compound 120); control generation of this fibroblast growth protein within the population of insects 100 via application of a stressor (e.g., chemical, biological, environmental stressors); and to extract and purify the fibroblast growth protein from other organic matter in the population of insects 100.

More specifically, a genome of the population of insects 100 can be genetically-modified to include: a target sequence 112 encoding for the fibroblast growth protein (i.e., the recombinant protein); a promoter sequence 114 coupled to the target sequence 112 and associated with the particular stressor, such that application of the stressor—such as a temperature stressor (e.g., heat-shock or cold-shock), a UV stressor (e.g., exposure to UV light), a nutrient stressor (e.g., nutrient-deficiency)—to the population of insects 100 triggers expression of the promoter sequence 114 in the population of insects 100, thereby triggering transcription of the downstream target sequence 112, leading to expression and therefore production of the fibroblast growth protein. Therefore, the method S100 can be executed to induce production of large quantities of fibroblast growth protein via the genetically-modified population of insects 100.

Further, in this implementation, the fibroblast growth factor can be mixed with a volume of basal media—including a set of ingredients matched to the fibroblast growth factor—to generate a volume of a growth media.

### 13.4 Example: Prolactin

In another implementation, a population of insects 100 can be genetically modified to generate a prolactin protein. In particular, human prolactin is a hormone configured for implementation as: a food ingredient that may be present in physiologically comparable concentrations in infant formula; and/or a processing aid configured to induce expression of milk production in human breast cells and/or induce proliferation/survival in oligodendrocytes, pancreatic stem cells etc.

Generally, in this implementation, Blocks of the method S100 can be executed to: cultivate a genetically-modified population of insects 100 configured to generate a prolactin protein (i.e., the target compound 120); control generation of this prolactin protein within the population of insects 100 via application of a stressor (e.g., chemical, biological, environmental stressors); and to extract and purify the prolactin protein from other organic matter in the population of insects 100.

More specifically, a genome of the population of insects 100 can be genetically-modified to include: a target sequence 112 encoding for the prolactin protein (i.e., the recombinant protein); a promoter sequence 114 coupled to the target sequence 112 and associated with the particular stressor, such that application of the stressor—such as a temperature stressor (e.g., heat-shock or cold-shock), a UV stressor (e.g., exposure to UV light), a nutrient stressor (e.g., nutrient-deficiency)—to the population of insects 100 triggers expression of the promoter sequence 114 in the population of insects 100, thereby triggering transcription of the downstream target sequence 112, leading to expression and therefore production of the prolactin protein. Therefore, the method S100 can be executed to induce production of large quantities of prolactin protein via the genetically-modified population of insects 100.

### 13.5 Example: Insulin

In one implementation, a population of insects 100 (e.g., a population of Drosophila) can be genetically modified to generate an insulin protein. Generally, in this implementation, Blocks of the method S100 can be executed to: cultivate a genetically-modified population of insects 100 configured to generate an insulin protein (i.e., the target compound 120); control generation of this insulin protein within the population of insects 100 via application of a stressor (e.g., chemical, biological, environmental stressors); and to extract and purify the insulin protein from other organic matter in the population of insects 100.

More specifically, a genome of the population of insects 100 can be genetically-modified to include: a target sequence 112 encoding for the insulin protein (i.e., the recombinant protein); a promoter sequence 114 coupled to the target sequence 112 and associated with the particular stressor, such that application of the stressor—such as a temperature stressor (e.g., heat-shock or cold-shock), a UV stressor (e.g., exposure to UV light), a nutrient stressor (e.g., nutrient-deficiency)—to the population of insects 100 triggers expression of the promoter sequence 114 in the population of insects 100, thereby triggering transcription of the downstream target sequence 112, leading to expression and therefore production of the insulin protein. Therefore, the method S100 can be executed to induce production of large quantities of insulin protein via the genetically-modified population of insects 100.

The resulting quantity of insulin protein can be stored as an insulin-enriched bioprocessing intermediate, which can then be further purified and transformed into an insulin product, such as insulin that can be injected or inhaled by human users for treatment of diabetes. Therefore, purity of the resulting quantities of insulin protein can be controlled in order to enable generation of pharmaceutical grade insulin from the insulin protein harvested from the population of insects 100.

Further, by genetically modifying insects to produce insulin protein—rather than rely on transgenic bacteria (e.g., E. Coli) or other types of prokaryotes—fully functional insulin protein (e.g., bioactive insulin protein) can be harvested directly from the population of insects 100, thereby eliminating additional processes—which result in excess waste, increased energy consumption, and increased production costs—traditionally required to functionalize insulin protein after production, thereby enabling high-throughput and low-cost production of functional insulin protein (e.g., bioactive insulin protein) via a highly sustainable process.

In this implementation, the method S100 can include: genetically modifying a population of insects 100 to produce an insulin protein in Block S102; during a growth period succeeding the initial period, cultivating the population of insects 100 according to a set of growth conditions in Block S110; and, during a treatment period succeeding the growth period, applying a first dosage of a first stressor to the population of insects 100, the first stressor configured to trigger production of the insulin protein by the population of insects 100 in Block S120. The method S100 further includes, in response to a first proportion of the insulin protein within the population of insects 100 exceeding a threshold proportion: harvesting the population of insects 100 in Block S130; homogenizing the population of insects 100 to form a blend including the first proportion of the insulin protein and a second proportion of a set of secondary components in Block S140; and separating the first proportion of the first target compound 120 from the second proportion of the set of secondary components in Block S150.

### 13.5.1 Genetic Modification

A population of insects 100 can be genetically modified to produce the insulin protein. In particular, insect cells can be genetically modified to include a target sequence 112 (e.g., an insulin encoding-sequence) encoding for the insulin protein, such that when the target sequence 112 is expressed, the insect cells generate the insulin protein.

As described above, the population of insects 100 can be genetically modified to include a set of regulatory sequences configured to control production of the insulin protein (i.e., target compound 120) expressed by the target sequence 112. In one implementation, the population of insects 100 can be genetically-modified to produce an insulin protein responsive to exposure to a first stressor (e.g., a heat stressor, a UV-light stressor, a chemical stressor), a genome of the population of insects 100 including: a first promoter sequence 114 configured to activate responsive to exposure to the first stressor; and a first target sequence 112 linked to (e.g., downstream) the first promoter sequence 114 and corresponding to (e.g., encoding for) the insulin protein.

For example, the population of insects 100 can be configured to include the promoter sequence 114: upstream the target sequence 112; and associated with a particular stressor (e.g., heat-shock, cold-shock, nutrient-deprivation, dehydration, UV exposure), such that application of the particular stressor to the population of insects 100 induces expression of the promoter sequence 114, thereby inducing expression of the target sequence 112 and thus expression of the insulin protein in the population of insects 100. Therefore, the particular stressor—associated with the promoter sequence 114—can be applied to the population of insects 100 to induce and/or promote increased production of the insulin protein within the population of insects 100.

Additionally, in another example, the population of insects 100 can be configured to include a leader sequence 116 upstream the target sequence 112 and downstream the promoter sequence 114, thereby enabling translation of the target sequence 112 during application of the particular stressor.

The population of insects 100 can be genetically modified to express the insulin protein in specific tissues within insects in the population of insects 100. In particular, the population of insects 100 can be genetically modified to express the insulin protein within a particular tissue configured to enable an increased rate of insulin protein production and increased functionality of the resulting insulin protein. Further, this tissue can be selected based on a target structure and/or target functionality of the resulting insulin protein.

For example, a first population of insects 100 can be genetically modified to include a first set of transgenic sequences—configured to be transcribed within a first type of tissue within the first population of insects 100, the first set of transgenic sequences including: a target sequence 112 encoding for the insulin protein; a promoter sequence 114 coupled to the target sequence 112 and associated with a particular stressor (e.g., a temperature stressor, a UV stressor, a nutrient stressor), such that expression of the promoter sequence 114, in the first type of tissue, triggers transcription of the downstream target sequence 112 within the first type of tissue, leading to expression and therefore production of the insulin protein—characterized by a first functionality—in the first type of tissue at a first production rate. Additionally and/or alternatively, in this example, a second population of insects 100 can be genetically modified to include the first set of transgenic sequences—configured to be transcribed within a second type of tissue within the second population of insects 100, such that expression of the promoter sequence 114, in the second type of tissue, triggers transcription of the downstream target sequence 112 within the second type of tissue, leading to expression and therefore production of the insulin protein—characterized by a second functionality less than the first functionality—in the second type of tissue at a second production rate less than the first rate.

Therefore, the population of insects 100 can be genetically modified to selectively produce the insulin protein within a particular tissue in order to promote increased rate of insulin production within the population of insects 100 and/or increased functionality of the insulin protein, thereby minimizing additional processing for increasing this functionality.

Additionally, in one variation, the population of insects 100 can be genetically modified to include an affinity tag (e.g., a peptide sequence)—linked to the target sequence 112—configured to enable purification of the resulting insulin protein, such as via affinity chromatography.

### 14.3.1.1 Genetic Modification: Multiple Generations

The population of insects 100 can define a genetically-modified line of insects including multiple generations. In this implementation, a first generation of the population of insects 100 can be genetically modified to produce the insulin protein. This genome can then be propagated through the line of insects, over multiple generations, without further genetic modification, as described above.

In one variation, the population of insects 100 can be genetically modified to include a set of regulatory sequences configured to promote stability of the target sequence 112—and/or other transgenic sequences such as the promoter sequence 114, the leader sequence 116, and/or the reporter sequence—within the genome of the population of insects 100 over multiple generations. In particular, in one implementation, the population of insects 100 can be genetically modified to include a set of balancer chromosomes configured to enable propagation of the target sequence 112 and/or other transgenic sequences in the genome of the population of insects 100 between successive generations.

For example, a first generation of a population of Drosophila can be genetically modified to include: a target sequence 112 encoding for the insulin protein; and a regulatory sequence (e.g., a balancer chromosome)—encoding for a curly-wing trait—coupled to the target sequence 112 and configured to maintain the target sequence 112 within the genomes of successive generations of the population of Drosophila, such as by guarding against changes or mutations in segments of the genome containing the target sequence 112 between generations of the population of Drosophila. In this example, presence of the target sequence 112 within successive generations of the population of insects 100 can be confirmed via visual detection of curly wings in the population of Drosophila. Further, by genetically modifying the population of Drosophila to include the curly-wing trait and therefore include curly wings—which are distinct from wild-type Drosophila—containment and handling of the population of Drosophila is simplified due to the inability of Drosophila with curly wings to fly.

### 13.5.2 Insulin Production

The population of insects 100 can be cultivated during a growth period according to a set of growth conditions—such as a particular temperature range, duration of the growth period, and/or nutrient source—configured to increase a survival rate of insects in the population of insects 100 and therefore promote increased production of the insulin protein, as described above.

In one implementation, the population of insects 100 can be cultivated according to the set of growth conditions corresponding to generation of the insulin protein. In particular, the set of growth conditions can be tailored to generation of the insulin protein in order to maximize a rate of insulin production within the population of insects 100. For example, during a first growth period, a first population of insects 100 can be cultivated according to a first set of growth conditions including incubation of the first population of insects 100 at a first temperature for a first duration. Then, during a first treatment period succeeding the first growth period, the first population of insects 100 can be subjected to a stressor—according to a first protocol—configured to control enrichment (e.g., unit mass of insulin protein per unit mass of insects) of the target compound 120 within the first population of insects 100. Upon completion of the first treatment period, the first population of insects 100 can be harvested for extraction of a first quantity of the insulin protein from the first population of insects 100. Additionally, during a second growth period, a second population of insects 100 can be cultivated according to a second set of growth conditions including incubation of the second population of insects 100 at a second temperature greater than the first temperature for the first duration. Then, during a second treatment period succeeding the second growth period, the second population of insects 100 can be subjected to the stressor according to the first protocol. Upon completion of the second treatment period, the second population of insects 100 can be harvested for extraction of a second quantity of the insulin protein from the second population of insects 100. Then, in response to the second quantity exceeding the first quantity of the insulin protein, a third population of insects 100 can be cultivated according to a third set of growth conditions including incubation of the third population of insects 100 at a third temperature exceeding the second temperature. This process can be repeated to converge on a set of growth conditions that yield a highest quantity of insulin protein.

The population of insects 100 can then be subjected to a particular stressor during a treatment period in order to promote expression of the insulin protein within the population of insects 100. Similarly, the particular stressor and/or application of the particular stressor can be tailored to generation of the insulin protein in order to maximize a rate of insulin production within the population of insects 100. Additionally, the treatment period can include a set of treatment cycles (e.g., one treatment cycle, two treatment cycles, ten treatment cycles) in order to increase enrichment of the insulin protein within the population of insects 100.

### 13.5.3 Purification of Insulin

Upon completion of the treatment period, the population of insects 100 can be harvested for collection of the insulin protein. In particular, the population of insects 100 can be euthanized before mechanically homogenizing the population of insects 100 in a buffer solution to form an insulin blend (e.g., a homogenous mixture) including an amount of the insulin protein and an amount of secondary components. The buffer solution can be matched to the insulin protein, such that the buffer solution promotes functionality of the insulin protein.

The insulin blend can then be further filtered to remove insoluble materials from the insulin blend to form an insulin mixture (e.g., a liquid insulin mixture) including insulin proteins and insect proteins in the buffer solution.

This insulin mixture can then be stored—under particular conditions—for further processing and generation of insulin products (e.g., injectable insulin). Alternatively, the insulin mixture can be further purified to remove insect proteins from the insulin mixture. For example, as described above, contaminants—including insect protein—can be removed from the insulin mixture via size exclusion chromatography, ion exchange chromatography, affinity chromatography (e.g., via inclusion of an affinity tag coupled to the target sequence 112), and/or TPP.

### 13.5.4 Storage & Shelf-Life

The resulting insulin-mixture can then be stored within particular conditions in order to maintain functionality of the insulin protein within the insulin mixture, thereby extending a shelf-life of the insulin mixture. For example, the insulin mixture—including a liquid mixture of insulin protein and drosophila protein—can be stored (e.g., frozen) within a particular temperature range (e.g., less than −20 degrees Celsius) configured to maintain functionality of the insulin protein within the liquid insulin mixture.

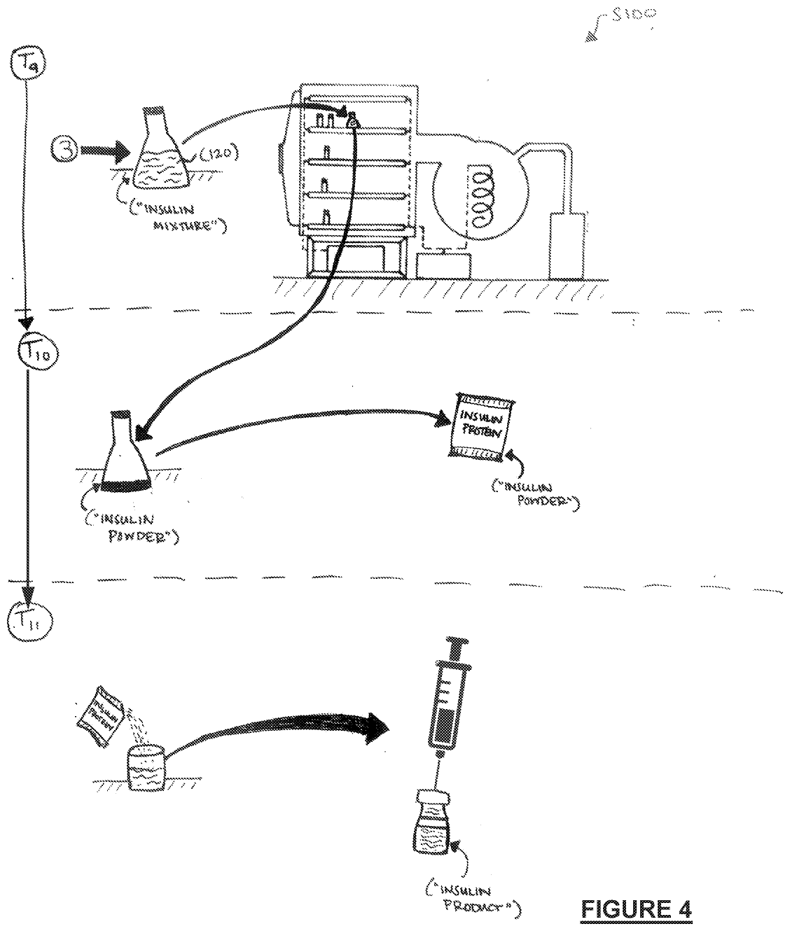

In one variation, as shown in FIG. 4, the resulting insulin mixture can be further processed in order to extend a shelf-life of the insulin mixture. For example, the insulin mixture can be freeze-dried (e.g., via lyophilization)—according to a particular method, such as within a particular temperature range, over a particular duration, and/or in a particular buffer solution—to form an insulin powder. The resulting insulin powder can therefore be readily stored and/or shipped—without requiring refrigeration or freezing—and maintain functionality of the insulin protein. The insulin powder can then be reconstituted—and/or further processed according to various pharmaceutical techniques—in order to generate an insulin product (e.g., inhalable insulin, injectable insulin).

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method comprising:
during an initial period, genetically modifying a population of Drosophila to produce a first target compound responsive to exposure to a heat stressor, a genome of the population of Drosophila comprising:
a first promoter sequence configured to activate responsive to exposure to the heat stressor; and
a first target sequence linked to the first promoter sequence and corresponding to the first target compound;
during a growth period succeeding the initial period, cultivating the population of Drosophila for a first duration under a first set of growth conditions;
during a treatment period succeeding the growth period:
during a first treatment cycle within the treatment period, applying a first dosage of the heat stressor to the population of Drosophila to trigger production of the first target compound in the population of Drosophila at a first rate proportional the first dosage;
during a second treatment cycle succeeding the first treatment cycle within the treatment period, applying a second dosage of the heat stressor, the second dosage greater than the first dosage, to the population of Drosophila to trigger production of the first target compound at a second rate greater than the first rate and proportional the second dosage; and
based on a first predicted amount of the first target compound produced in the population of Drosophila during the treatment period, harvesting the population of Drosophila; and
during a purification period succeeding the treatment period, in response to harvesting the population of Drosophila:
homogenizing the population of Drosophila to generate a blend comprising secondary components and a first amount of the first target compound; and
extracting the first amount of the first target compound from the blend.

2. The method of claim 1, wherein genetically modifying the population of Drosophila to produce the first target compound responsive to exposure to the heat stressor comprises genetically modifying the population of Drosophila to produce the first target compound, comprising an insulin protein, responsive to exposure to the heat stressor, the genome comprising:
the first promoter sequence configured to activate responsive to exposure to the heat stressor; and
the first target sequence linked to the first promoter sequence and corresponding to the insulin protein.

3. The method of claim 1, wherein genetically modifying the population of Drosophila to produce the first target compound responsive to exposure to the heat stressor comprises genetically modifying the population of Drosophila to produce the first target compound, comprising a fibroblast growth factor, responsive to exposure to the heat stressor, the genome comprising:
the first promoter sequence configured to activate responsive to exposure to the heat stressor; and
the first target sequence linked to the first promoter sequence and corresponding to the fibroblast growth factor.

4. The method of claim 3, further comprising, mixing the amount of the first growth factor with a first volume of a basal media matched to the first growth factor to generate a second volume of a growth media.

5. The method of claim 1:
wherein extracting the first amount of the first target compound from the blend comprises extracting the first amount of the first target compound from the blend during an extraction period; and
further comprising, during a processing period succeeding the extraction period:
mixing the first amount of the first target compound with a volume of a buffer solution to generate a first mixture;
lyophilizing the first mixture to generate a lyophilized mixture comprising the first amount of the first target compound; and
storing the lyophilized mixture according to a set of storage conditions corresponding to the target compound.

6. The method of claim 1:
wherein genetically modifying the population of Drosophila to produce the first target compound responsive to exposure to the heat stressor comprises genetically modifying the population of Drosophila to produce the first target compound responsive to exposure to the heat stressor, the genome comprising:
the first promoter sequence configured to activate responsive to exposure to the heat stressor and associated with a first tissue type in a set of tissue types; and
the first target sequence linked to the first promoter sequence and corresponding to the first target compound;
wherein applying the first dosage of the heat stressor to the population of Drosophila to trigger production of the first target compound in the population of Drosophila comprises applying the first dosage of the heat stressor to the population of Drosophila to trigger production of the first target compound in tissues of the first tissue type in the population of Drosophila; and
wherein applying the second dosage of the heat stressor to the population of Drosophila to trigger production of the first target compound in the population of Drosophila comprises applying the second dosage of the heat stressor to the population of Drosophila to trigger production of the first target compound in tissues of the first tissue type in the population of Drosophila.

7. The method of claim 1:
wherein cultivating the population of Drosophila during the growth period comprises cultivating the population of Drosophila during the growth period spanning a transition of the population of Drosophila from an embryonic stage to a larval stage; and
wherein harvesting the population of Drosophila comprises harvesting the population of Drosophila in the larval stage.

8. The method of claim 1:
wherein genetically modifying the population of Drosophila to produce the first target compound responsive to exposure to the heat stressor comprises genetically modifying the population of Drosophila to produce the first target compound and a second target compound responsive to exposure to the heat stressor, the genome comprising:
the first promoter sequence configured to activate responsive to exposure to the heat stressor;
the first target sequence linked to the first promoter sequence and corresponding to the first target compound; and
a second target sequence linked to the first promoter sequence and corresponding to the second target compound;
wherein applying the first dosage of the heat stressor to the population of Drosophila to trigger production of the first target compound in the population of Drosophila comprises applying the first dosage of the heat stressor to the population of Drosophila to:
trigger production of the first target compound in the population of Drosophila at the first rate proportional the first dosage; and
trigger production of the second target compound in the population of Drosophila at a third rate proportional the first dosage;
wherein applying the second dosage of the heat stressor to the population of Drosophila to trigger production of the first target compound in the population of Drosophila comprises applying the second dosage of the heat stressor to the population of Drosophila to:
trigger production of the first target compound in the population of Drosophila at the second rate proportional the second dosage; and
trigger production of the second target compound in the population of Drosophila at a fourth rate proportional the second dosage, the fourth rate exceeding the second rate;
wherein harvesting the population of Drosophila based on the first predicted amount of the first target compound produced in the population of Drosophila during the treatment period comprises, based on the on the first predicted amount of the first target compound and a second predicted amount of the second target, harvesting the population of Drosophila;
wherein homogenizing the population of Drosophila to generate the blend comprising secondary components and the first amount of the first target compound comprises homogenizing the population of Drosophila to generate the blend comprising secondary components, the first amount of the first target compound, and a second amount of the second target compound; and
further comprising, extracting the second amount of the second target compound from the blend.

9. The method of claim 8, wherein genetically modifying the population of Drosophila to produce the first target compound and the second target compound comprises genetically modifying the population of Drosophila to produce the first target compound and the second target compound, the genome comprising:
the first promoter sequence configured to activate responsive to exposure to the heat stressor;
a first leader sequence linked to the first promoter sequence and configured to:
regulate translation of the first target sequence; and
regulate translation of the second target sequence;
the first target sequence linked to the first leader sequence and the first promoter sequence and corresponding to the first target compound; and
the second target sequence linked to the first leader sequence and the first promoter sequence and corresponding to the second target compound.

10. The method of claim 1:
wherein genetically modifying the population of Drosophila to produce the first target compound comprises genetically modifying the population of Drosophila to produce the first target compound according to a target set of characteristics comprising a target structure and a target functionality, the genome comprising:
the first promoter sequence configured to:
activate responsive to exposure to the heat stressor; and
trigger transcription of the first target sequence to trigger production of the first target compound according to the target structure and the target functionality; and
the first target sequence linked to the first promoter sequence and corresponding to the first target compound; and
wherein extracting the first amount of the first target compound from the blend comprises extracting the first amount of the first target compound from the blend, the first target compound exhibiting the target structure and the target functionality.

11. The method of claim 1:
wherein cultivating the population of Drosophila for the first duration under the first set of growth conditions comprises cultivating the population of Drosophila for the first duration under the first set of growth conditions, the first set of growth conditions comprising a first temperature range;
wherein applying the first dosage of the heat stressor to the population of Drosophila comprises locating the population of Drosophila within a treatment chamber at temperatures within a second temperature range, temperatures within the second temperature range exceeding temperatures within the first temperature range; and
wherein applying the second dosage of the heat stressor to the population of Drosophila comprises locating the population of Drosophila within the treatment chamber at temperatures within a third temperature range, temperatures within the third temperature range exceeding temperatures within the second temperature range.

12. The method of claim 1, wherein harvesting the population of Drosophila based on the first predicted amount of the first target compound produced in the population of Drosophila during the treatment period comprises:
predicting a first concentration of the first target compound produced within the population of Drosophila during the treatment period; and
in response to the first concentration exceeding a threshold concentration defined for the treatment period, harvesting the population of Drosophila.

13. The method of claim 12, wherein harvesting the population of Drosophila in response to the first concentration exceeding the threshold concentration comprises harvesting the population of Drosophila in response to the first concentration exceeding the threshold concentration of one percent.

14. A method comprising:
during an initial period, modifying a population of Drosophila to produce a first target compound, in a set of target compounds, a genome of the population of Drosophila comprising:
a promoter sequence inducible by a first stressor and associated with cell tissue of a first tissue type; and
a first target sequence coupled to the first promoter sequence and corresponding to the first target compound;
during a growth period succeeding the initial period, cultivating the population of Drosophila according to a set of growth conditions;
during a treatment period succeeding the growth period, applying a first dosage of the first stressor to the population of Drosophila to activate the first promoter sequence and trigger production of the first target compound in tissue of the first tissue type in the population of Drosophila;
during a harvest period succeeding the treatment period:
harvesting the population of Drosophila;
homogenizing the population of Drosophila to form a blend comprising a set of secondary components and a first amount of the first target compound;
extracting the first amount of the first target compound from the blend; and
mixing the first amount of the first target compound with a set of stabilizing agents configured to stabilize the first target compound to generate a compound mixture; and
during a storage period, storing the compound mixture according to a set of storage conditions defined for the first target compound.

15. The method of claim 14:
wherein extracting the first amount of the first target compound from the blend comprises extracting the first amount of the first target compound from the blend, the first target compound characterized by a first functionality corresponding to the first tissue type;
wherein mixing the first amount of the first target compound with the set of stabilizing agents to generate the compound mixture comprises mixing the first amount of the first target compound with the set of stabilizing agents to generate the compound mixture, the set of stabilizing agents configured to promote the first functionality and stabilize the first target compound within the compound mixture;
wherein storing the compound mixture according to the set of storage conditions defined for the first target compound comprises storing the compound mixture according to the set of storage conditions defined for the first target compound and the first functionality; and
further comprising:
during a first time period, modifying a second population of Drosophila to produce the first target compound, a second genome of the second population of Drosophila comprising:
a second promoter sequence inducible by the first stressor and associated with cell tissue of a second tissue type; and
the first target sequence coupled to the second promoter sequence and corresponding to the first target compound;
during a second growth period succeeding the first time period, cultivating the second population of Drosophila according to the set of growth conditions;
during a second treatment period succeeding the second growth period, applying a second dosage of the first stressor to the second population of Drosophila to activate the second promoter sequence and trigger production of the first target compound in tissue of the second tissue type in the second population of Drosophila;
during a second harvest period succeeding the second treatment period:
harvesting the second population of Drosophila;
homogenizing the second population of Drosophila to form a second blend comprising a second set of secondary components and a second amount of the first target compound;
extracting the second amount of the first target compound from the second blend, the first target compound characterized by a second target functionality corresponding to the second tissue type; and
mixing the second amount of the second target compound with a second set of stabilizing agents to generate a second compound mixture, the second set of stabilizing agents configured to promote the second functionality and stabilize the first target compound within the second compound mixture; and
during a second storage period, storing the second compound mixture according to a second set of storage conditions defined for the first target compound and the second functionality.

16. The method of claim 14:
wherein modifying the population of Drosophila to produce the first target compound comprises modifying the population of Drosophila to produce the first target compound, a genome of the population of Drosophila comprising:
the promoter sequence inducible by a heat stressor and associated with cell tissue of a first tissue type; and
the first target sequence coupled to the first promoter sequence and corresponding to the first target compound; and
wherein applying the first dosage of the first stressor to the population of Drosophila to activate the first promoter sequence and trigger production of the first target compound in tissue of the first tissue type in the population of Drosophila comprises applying the first dosage of the heat stressor to the population of Drosophila to activate the first promoter sequence and trigger production of the first target compound in tissue of the first tissue type in the population of Drosophila.

17. The method of claim 14:
wherein modifying the population of Drosophila to produce the first target compound comprises modifying the population of Drosophila to produce the first target compound, a genome of the population of Drosophila comprising:
the promoter sequence inducible by a chemical stressor and associated with cell tissue of a first tissue type; and
the first target sequence coupled to the first promoter sequence and corresponding to the first target compound; and
wherein applying the first dosage of the chemical stressor to the population of Drosophila to activate the first promoter sequence and trigger production of the first target compound in tissue of the first tissue type in the population of Drosophila comprises applying the first dosage of the heat stressor to the population of Drosophila to activate the first promoter sequence and trigger production of the first target compound in tissue of the first tissue type in the population of Drosophila.

18. The method of claim 14:
wherein applying the first dosage of the first stressor to the population of Drosophila to activate the first promoter sequence and trigger production of the first target compound in tissue of the first tissue type in the population of Drosophila comprises, during a first treatment cycle within the treatment period, applying the first dosage of the first stressor to the population of Drosophila to activate the first promoter sequence and trigger production of the first target compound, at a first rate proportional the first dosage, in tissue of the first tissue type in the population of Drosophila; and
further comprising, during a second treatment cycle succeeding the first treatment cycle within the treatment period, applying a second dosage, exceeding the first dosage, of the first stressor to the population of Drosophila to activate the first promoter sequence and trigger production of the first target compound, at a second rate greater than the first rate and proportional the second dosage, in tissue of the first tissue type in the population of Drosophila.

19. A method comprising:
during a first production cycle a first population of Drosophila genetically-modified to produce a first target compound:
during a first growth period, cultivating the first population of Drosophila according to a first set of growth conditions, a first genome of the first population of Drosophila comprising:
a first promoter sequence associated with a first stressor and corresponding to a first set of target characteristics of the first target compound; and
a first target sequence coupled to the first promoter sequence and corresponding to the first target compound;
during a first treatment period succeeding the first growth period, applying a first dosage the first stressor to the population of Drosophila to activate the first promoter sequence and trigger production of the first target compound in the first population of Drosophila; and
during a first harvest period succeeding the first treatment period:
harvesting the first population of Drosophila;
homogenizing the first population of Drosophila to form a first blend comprising a first set of secondary components and a first amount of the first target compound; and
extracting the first amount of the first target compound, exhibiting the first set of target characteristics, from the first blend; and
during a second production cycle for a second population of Drosophila genetically-modified to produce a second target compound:
during a second growth period, cultivating the second population of Drosophila according to a second set of growth conditions, a second genome of the second population of Drosophila comprising:
a second promoter sequence associated with a second stressor and corresponding to a second set of target characteristics of the second target compound; and
a second target sequence coupled to the second promoter sequence and corresponding to the second target compound;
during a second treatment period succeeding the second growth period, applying a second dosage of the second stressor to the population of Drosophila to activate the second promoter sequence and trigger production of the second target compound in the second population of Drosophila; and
during a second harvest period succeeding the second treatment period:
harvesting the second population of Drosophila;
homogenizing the second population of Drosophila to form a second blend comprising a second set of secondary components and a second amount of the second target compound; and
extracting the second amount of the second target compound, exhibiting the second set of target characteristics, from the second blend.

20. The method of claim 19, further comprising:
during the first production cycle:
mixing the first amount of the first target compound with a first set of stabilizing agents configured to stabilize the first target compound to generate a first compound mixture; and
storing the first compound mixture according to a first set of storage conditions defined for the first target compound; and
during the second production cycle:
mixing the second amount of the second target compound with a second set of stabilizing agents configured to stabilize the second target compound to generate a second compound mixture; and
storing the second compound mixture according to a second set of storage conditions defined for the second target compound.
